(19) United States
(12) Patent Application Publication
(10) Pub. No.: US 2015/0258043 A1
(43) Pub. Date: Sep. 17, 2015

(54) USE OF SPHINGOID LONG CHAIN BASES AND THEIR ANALOGS IN TREATING AND PREVENTING BACTERIAL INFECTIONS

(71) Applicants: YEDA RESEARCH AND DEVELOPMENT CO. LTD., Rehovot (IL); UNIVERSITY OF DUISBURG-ESSEN, Essen (DE)

(72) Inventors: Anthony Futerman, Rehovot (IL); Yael Pewzner-Jung, Rehovot (IL); Erich Gulbins, Essen (DE)

(21) Appl. No.: 14/435,848

(22) PCT Filed: Oct. 15, 2013
(86) PCT No.: PCT/IL2013/050831

§ 371 Date: Apr. 15, 2015

Related U.S. Application Data
(60) Provisional application No. 61/713,653, filed on Oct. 15, 2012.

(51) Int. Cl. A61K 31/133; A61K 31/7028; A61K 9/00; A61K 31/225; A61K 31/661; A61K 31/662; A61K 31/137; A61K 31/164

(52) U.S. Cl. CPC A61K 31/133; A61K 31/137; A61K 31/7028; A61K 31/164; A61K 31/225; A61K 31/661; A61K 31/662; A61K 9/0073

(57) ABSTRACT

Methods of preventing or treating bacterial infections, including but not limited to Pseudomonas aeruginosa infections, by administering sphingoid long chain bases (LCB) are provided. The invention further relates to the use of sphingoid long chain bases, or pharmaceutical composition comprising same, for the treatment of lung diseases or disorders such as cystic fibrosis and chronic obstructive pulmonary disease.

### FIELD OF THE INVENTION

The present invention relates to use of analogs of sphingoid long chain bases (LCB), and pharmaceutical compositions comprising same, in preventing and treating bacterial infection. The invention further relates to the prevention or treatment of lung diseases or disorders including but not limited to cystic fibrosis (CF) and chronic obstructive pulmonary disease (COPD).

### BACKGROUND OF THE INVENTION

Pseudomonas aeruginosa (P. aeruginosa) infections are of major clinical importance in patients with CF and chronic COPD, with ˜80% of CF patients suffering from chronic P. aeruginosa pneumonia by the age of 25, and one third of patients with COPD hosting the bacteria. P. aeruginosa infections are also of great importance in acute pneumonia in patients with trauma, burn wounds, sepsis or requiring ventilation. Therefore, it is of importance to identify susceptibility factors for P. aeruginosa pneumonia and to identify potential novel treatments. Enforcing antibacterial systems of the host to prevent infection would also circumvent the critical clinical problem of the high resistance of P. aeruginosa to almost all antibiotics.

Several studies indicate the importance of acid sphingomyelinase and its product ceramide in infection of mammalian cells with a variety of pathogens including P. aeruginosa, Staphylococcus aureus (S. aureus), Neisseria gonorrhoea, mycobacteria, Salmonella typhi, Listeria monocytogenes and E. coli. Further studies revealed that ceramide levels are increased in airway epithelial cells of patients with CF and that ceramide is an important mediator in pulmonary infection of CF patients with P. aeruginosa. However, it is unknown whether other sphingolipids, such as sphingosine, which is generated from ceramide by ceramidases, play a role in pulmonary infection, particularly in individuals with recurrent bacterial infections such as in CF.

International Patent Application No. WO 2008/006007 provides treatment of CF and P. aeruginosa infections by administering a therapeutic amount of one or more agents that inhibit de novo sphingolipid synthesis or recycling pathways.

US Patent Application No. 2005/0209219, to one of the inventors of the present invention, relates to the use of inhibitors of acid sphingomyelinase and/or inhibitors of products of the reaction catalyzed by said enzyme, for the prophylaxis and/or treatment of infectious diseases and/or diseases which are influenced by infections during the course thereof. Preferably, neutralizing antibodies and/or anti-depressants, especially tricyclic and/or tetracyclic and depressants, are used as inhibitors.

US Patent Application No. 2010/0285139 to one of the inventors of the present invention, relates to pharmaceutical composition for prophylaxis and/or treatment of infections and/or infectious diseases occurring in CF includes at least one antidepressant; at least one dispersant; and at least one pharmaceutically tolerable carrier material.

Wenderska, I. B. et al. (ChemBioChem 2011, 12, 2759-2766) discloses small molecules inhibitors of P. aeruginosa biofirm formation.

Bibel, D. J. et al. (J Invest Dermatol. 1992 March; 98(3):269-73) describes the antimicrobial activity of sphingosines against Staphylococcus aureus and other bacteria.

There exists a long-felt need for effective long-active means for preventing and curing infections, such as pathologies associated with P. aeruginosa infection.

### SUMMARY OF THE INVENTION

The present invention provides sphingoid long chain bases (LCB) and pharmaceutical compositions comprising same for use in preventing, ameliorating and treating bacterial infections, including but not limited to Pseudomonas aeruginosa (P. aeruginosa). The invention further provides methods of preventing or treating lung disorders such as CF and COPD by administering an effective amount of the LCBs described herein.

As demonstrated herein, LCBs such as sphingosine [(2S,3R)-2-aminooctadec-4-ene-1,3-diol; D-erythro spingosine] and its related derivatives dihydrosphingosine [sphinganine, (2S,3R)-2-aminooctadecane-1,3-diol; D-erythro-sphinganine] and 4-D-hydroxysphinganine (phytosphingosine), as well as a variety of non-naturally occurring LCBs, are effective in vitro and in vivo against bacterial infections, including but not limited to P. aeruginosa infections. These LCBs are therefore useful as anti-microbial agents, especially for treating lung infections, e.g., infections that are associated with CF and COPD. It has now unexpectedly been discovered that certain non-naturally occurring LCBs [e.g., D-threo, L-threo and L-erythro isomers of sphingosine or sphinganine, as well as longer carbon chain (e.g., C20) LCBs], are advantageous as anti-bacterial agents as compared to the naturally occurring D-erythro sphingosine and sphinganine analogs. Without wishing to be limited by any particular theory or mechanism of action, it is hypothesized that the non-natural sphingoid LCBs possess improved properties since they are metabolized to a lesser extent in mammals as compared with the natural analogs. As such, these compounds are expected to have improved drug-like properties, e.g., a better safety profile and longer half life as compared with the naturally occurring analogs. Furthermore, as demonstrated herein, preferred LCBs are those that have a carbon chain of 18 or longer, and preferably comprise a hydroxyl at C1 and C3, and an amine at C2. Finally, since the bacteria are negatively charged, LCBs that are positively charged in vivo are preferred.

Thus, according to one aspect, the present invention provides pharmaceutical composition comprising as an active ingredient a sphingoid long chain base (LCB) compound and a pharmaceutically acceptable carrier, for use in preventing or treating a bacterial infection, wherein the pharmaceutical composition is formulated for inhalation, and wherein the sphingoid LCB compound is represented by the structure of formula (I) or formula (II).

According to another aspect, the present invention provides method for preventing or treating a bacterial infection in a subject in need thereof, comprising the step of administering to the subject, by inhalation, a therapeutically effective amount of a pharmaceutical composition comprising a sphingoid long chain base (LCB) compound and a pharmaceutically acceptable carrier, wherein the sphingoid LCB compound is represented by the structure of formula (I) or formula (II).

The compounds of formula (I) and (II) are represented below:
(i) formula (I):

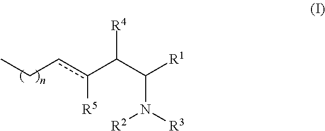

wherein
R¹ is H, CH₃, or —CH₂OR^{a} wherein R^{a} is H, C1-C4 alkyl or a sugar moiety;
R² and R³ are each selected from H or C1-C4 alkyl;
R⁴ and R⁵ are each independently H or OH;
n is an integer between 10 and 16; and
[symbol] is an optional double bond,
with the proviso that the following compounds are excluded:
   D-erythro-sphingosine;
   4-D-hydroxysphinganine (phytosphingosine);
   D-erythro-dihydrosphingosine (D-erythro-sphinganine);
   D,L-erythro-dihydrosphingosine (D,L-erythro-sphinganine); and
   D,L-threo-dihydrosphingosine (D,L-threo-sphinganine);
and salts, hydrates, solvates, polymorphs, optical isomers, enantiomers, diastereomers, epimers, and mixtures thereof.
(ii) formula (II):

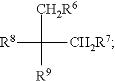

wherein
one of R⁶, R⁷ and R⁸ is NR^{b}R^{c}, and the other two are OR^{d}, wherein R^{b}, R^{c} and R^{d} are each H or C1-C4 alkyl;
R⁹ is a alkyl-phenyl wherein the phenyl is substituted by a C6-C20 alkyl group;
and salts, hydrates, solvates, polymorphs, optical isomers, enantiomers, diastereomers, epimers, and mixtures thereof.

In one embodiment, the compound is represented by the structure of formula (I). In some embodiments, the compound is a non-natural sphingoid LCB. In particular embodiments, the compound is selected from non-natural isomers of sphingosine, dihydrosphingosine and phytosphingosine. Each possibility represents a separate embodiment of the present invention.

In another embodiment, the compound is positively charged in vivo. In yet another embodiment, the compound is not a substrate of ceramide synthase.

In specific embodiments, the compound is selected from a D-threo, L-threo and L-erythro isomer of the compound of formula (I). In another embodiment, the sphingoid LCB comprises at least 18 carbons, at least 19 carbons, preferably at least 20 carbons. Each possibility represents a separate embodiment of the present invention.

In another embodiment, the compound is represented by the structure of formula (I), wherein R¹ is CH₂OH. In another embodiment, R¹ is selected from H, CH₃, CH₂OCH₃, —CH₂—O-galactosyl and —CH₂—O-glucosyl. Also, when present, the sugar may be a monosaccharide selected from a 5 carbon sugar (pentose) and 6 carbon sugar (hexose). Preferably the sugar is in ring form (furanose or pyranose). Each possibility representing a separate embodiment of the present invention.

In another embodiment, the compound is represented by the structure of formula (I), wherein R² and R³ are each H. In another embodiment, the compound is represented by the structure of formula (I), wherein R⁴ is OH and R⁵ is H.

In another embodiment, the compound is represented by the structure of formula III

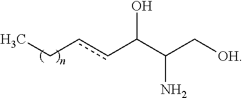

In some embodiments, the compound is represented by the structure of formula (I), wherein [symbol] is a double bond. In other embodiments [symbol] is a single bond.

In additional embodiments, the compound is represented by the structure of formula (II). In particular embodiments, one of R⁶, R⁷ and R⁸ is NH₂, and the other two are OH. In another embodiment, the compound of formula (II) is represented by the structure of formula IIa or IIb:

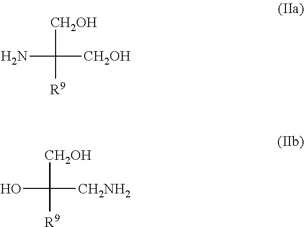

In particular embodiments, R⁹ is an alkyl-phenyl, wherein the phenyl is substituted by a C6-C10 alkyl, preferably by a C8 alkyl. In another particular embodiment, R⁹ is represented by the structure:

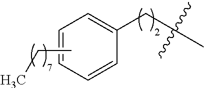

According to some embodiments, the LCB compound is a D-erythro isomer. According to additional embodiments, the LCB compound is a D-threo isomer. According to further embodiments, the LCB compound is a L-erythro isomer. According to yet additional embodiments, the LCB compound is a L-threo isomer.

According to particular embodiments, the LCB the compound is selected from the group consisting of: D-erythro-C20-sphingosine; D-threo-sphingosine; L-threo-sphingosine; L-erythro-sphingosine; D-threo-dihydrosphingosine (D-threo-sphinganine); L-threo-dihydrosphingosine (L-threo-sphinganine); L-erythro-dihydro sphingosine (L-erythro-sphinganine); 3-deoxy-D-erythro-sphingosine; 1-deoxy-D-erythro-dihydrosphingosine; 1-desoxymethylsphingosine; 1-deoxy-D-erythro-sphingosine; D-erythro-C16-sphingosine; Monomethyl D-erythro-sphingosine; Galactosyl D-erythro-sphingosine (psychosine); Glucosyl sphingosine; 2-amino-2-[2-(4-octylphenyl)ethyl]-1,3-propanediol (Fingolimod, FTY720); 2-(aminomethyl)-4-(4-octylphenyl)butane-1,2-diol; and 2-amino-2-(methoxymethyl)-4-(4-octylphenyl)butan-1-ol. Each possibility represents a separate embodiment of the present invention.

The bacterial infection may be caused by gram-positive bacteria or gram-negative bacteria, or a combination thereof, with each possibility representing a separate embodiment of the present invention. In a particular embodiment, said bacterial infection is caused by Pseudomonas, Staphylococcus aureus, Acinetobacter baumanii, Burkholderia species, or Mycobacteria species (typical and atypical). In one particular embodiment, the bacterial infection is a Pseudomonas infection. In another particular embodiment, said Pseudomonas infection is a Pseudomonas aeruginosa infection.

According to additional embodiments, said bacterial infection is a pulmonary bacterial infection. According to another embodiment, the subject having the bacterial infection is a subject having a lung disorder or disease. According to yet another embodiment, the lung disorder or disease is selected from the group consisting of cystic fibrosis (CF), chronic obstructive pulmonary disease (COPD) and pneumonia. Each possibility represents a separate embodiment of the present invention.

According to another embodiment, the subject is a mammal, particularly a human.

According to another embodiment, the present invention relates to a pharmaceutical composition comprising as an active ingredient a sphingoid long chain base (LCB) compound and a pharmaceutically acceptable carrier, for use in preventing or treating a lung bacterial infection, or a lung disorder or disease, wherein the pharmaceutical composition is formulated for inhalation, and wherein the sphingoid LCB compound is represented by the structure of formula (I′).

According to another embodiment, the present invention provides method for preventing or treating a bacterial infection in a subject in need thereof, comprising the step of administering to the subject, by inhalation, a therapeutically effective amount of a pharmaceutical composition comprising a sphingoid long chain base (LCB) compound and a pharmaceutically acceptable carrier, wherein the sphingoid LCB compound is represented by the structure of formula (I′)

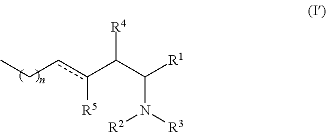

wherein
R¹ is H, CH₃, or —CH₂OR^{a} wherein R^{a} is H, C1-C4 alkyl or a sugar moiety;
R² and R³ are each selected from H or C1-C4 alkyl;
R⁴ and R⁵ are each independently H or OH;
n is an integer between 10 and 16; and
[symbol] is an optional double bond,
with the proviso that the following compounds are excluded:
   D-erythro-sphingosine;
   D-erythro-dihydrosphingosine (D-erythro-sphinganine);
and salts, hydrates, solvates, polymorphs, optical isomers, enantiomers, diastereomers, epimers, and mixtures thereof.

Compounds encompassed by formula (I′) include D-erythro-C20-sphingosine; D-threo-sphingosine; L-threo-sphingosine; L-erythro-sphingosine; D-threo-dihydro sphingosine (D-threo-sphinganine); L-threo-dihydrosphingosine (L-threo-sphinganine); L-erythro-dihydrosphingosine (L-erythro-sphinganine); 3-deoxy-D-erythro-sphingosine; 1-deoxy-D-erythro-dihydrosphingosine; 1 desoxymethylsphingosine; 1-deoxy-D-erythro-sphingosine; D-erythro-C16-sphingosine; Monomethyl D-erythro-sphingosine; Galactosyl D-erythro-sphingosine (psychosine); Glucosyl sphingosine; 4-D-hydroxysphinganine (phytosphingosine); D-erythro-dihydro sphingosine (D-erythro-sphinganine); L-erythro-dihydrosphingosine (L-erythro-sphinganine); D-threo-dihydrosphingosine (L-threo-sphinganine); L-threo-dihydro sphingosine (L-threo-sphinganine); 2-amino-2-[2-(4-octylphenyl)ethyl]-1,3-propanediol (Fingolimod, FTY720); 2-(aminomethyl)-4-(4-octylphenyl)butane-1,2-diol; and 2-amino-2-(methoxymethyl)-4-(4-octylphenyl)butan-1-ol. Each possibility represents a separate embodiment of the present invention.

These and other embodiments of the present invention will be better understood in relation to the figures, description, examples, and claims that follow.

### BRIEF DESCRIPTION OF THE FIGURES

FIGS. 1A-F. Immunohistochemical analysis demonstrates reduced sphingosine levels on airway epithelial cells of CF patients and in CF and CerS2 null mice. (A-D) Immunostaining of paraffin sections from of nasal tissues (A) healthy and CF individuals or trachea (B) and bronchi (C,D) of WT (upper row, C57BL/6 syngenic to CF; lower row, littermates of CerS2 null mice), CF and CerS2 null mice using a Cy3-coupled anti-sphingosine antibody reveals an almost complete lack of sphingosine in CF and CersS2 null cells, while sphingosine is clearly present in epithelia from healthy individuals or WT mice. (E,F) Ceramide accumulates in bronchial epithelial cells of CerS2 null and CF mice. Acid ceramidase (AC) inhalation restores sphingosine levels (C,D) and reduces ceramide (E,F) in CF and CerS2 null mice. Representative images are shown, together with fluorescence levels in arbitrary units (a.u.), (mean±s.d., n=5 in (A), n=4 in (B), n=6 for WT controls for CF mice (C57BL/6), n=8 for CF, and n=4 for all others in (C,D) and n=9 for untreated WT (C57BL/6) or CF, n=7 for AC inhaled WT, n=8 for AC inhaled CF and n=4 for CerS2 null mice in (E,F).

FIGS. 2A-D. Biochemical analysis confirms the reduction in sphingosine levels. (A) Total (left-hand panel) and surface sphingosine (middle, right-hand panel) is markedly reduced in tracheal epithelial cells of CF mice compared to WT, determined by a sphingosine kinase assay of total extracts from freshly isolated epithelial cells (left-hand panel), an in situ kinase assay on the tracheal surface (middle panel) or by immunoprecipitation of sphingosine (sphingosine-IP) from the luminal surface followed by quantification using a sphingosine kinase assay (right-hand panel). Incubation of the tracheal surface in vitro with acid ceramidase (AC) restores surface sphingosine levels (middle panel). Pre-incubation of WT trachea with cytochalasin B (CTB) demonstrates that blocking endocytosis does not change the results of the surface kinase assay or the immunoprecipitation studies (middle and right-hand panel). (B,C) Inhalation of 200 units acid ceramidase restores sphingosine (B) and reduces ceramide (C) levels in isolated tracheal epithelial cells of CF mice. Sphingosine (Sph) inhalation restores sphingosine levels in CF mice (B, right-hand panel). Incubation of trachea in vitro with acid ceramidase for 30 min also reduced surface ceramide in CF mice (C, right-hand panel). Sphingosine and ceramide were measured on the surface by an in situ kinase assay (B and C middle and right-hand panel) or in extracts of freshly-isolated epithelial cells (c, left-hand panel). (D) Acidification of the surface of CF trachea reduces surface ceramide and increases sphingosine levels. Data are means±s.d., n=4. (E) Specificity of the anti-sphingosine antibody. Upper panel, CF or WT mice trachea incubated in vitro with dihydrosphingosine (sphinganine), fixed and stained with the anti-sphingosine antibody. Lower panel, a representative autoradiogram of a TLC plate from 4 independent experiments indicating that the antibody does not immunoprecipitate sphinganine.

FIGS. 3A-D. Acid ceramidase, sphingosine or FTY720 inhalation rescue CF and CerS2 null mice from pulmonary P. aeruginosa infection. (A,B) The number of bacteria in the lungs and the overall sickness score (C,D) of WT, CerS2 null and CF mice 3-4 h after intranasal infection with 1×10⁸ CFU of P. aeruginosa strains 762 (A,C) and CF mice with strains PA14 and ATCC 27853. Mice were inhaled with 0.9% NaCl or acid ceramidase 1 h prior (b.i.) to infection, or with sphingosine or FTY720 1 h before (b.i.) or 1 h after (a.i.) infection. Sickness score 4, severely affected (unresponsive animal); score 3 affected, (heavy breathing, low body temperature, ruffled fur) score 2, moderately affected (ruffled fur, heavy breathing); score 1, slightly affected (ruffled fur); score 0, unaffected (healthy appearance). Data are means±s.d., n=4.

FIGS. 4A-D. The ceramide/sphingosine ratio regulates P. aeruginosa growth. (A) Representative image of two independent experiments (left hand panel) after addition of Sytox green (1 μM) for 30 min in the absence or presence of 50 μM sphingosine or 50 μM C16-ceramide (C16-Cer) in 1% DMSO. Fluorescent and thus permeabilised bacteria were quantified (right hand panel). (B-D) The inhibitory effect of sphingosine on the growth of P. aeruginosa strain 762 is decreased in vitro and in vivo by C16-ceramide. (B) Bacteria were incubated with 10 μM sphingosine, and increasing concentrations of C16-ceramide, and growth was determined by counting colony forming unit (CFU) after 24 hrs growth on LB agar plates. Number of bacteria in the lung (C) and sickness score (D) of CF mice or WT mice inhaled with either sphingosine or a combination of sphingosine and C16-ceramide 1 h before (b.i.) with 10⁸ P. aeruginosa CFU strain 762. Data are means±s.d., n=3 in (A,B) and n=4 in (C,D).

FIG. 5. Trachea and bronchi from Cers2 null mice accumulate ceramide. Staining of paraffin sections from trachea and bronchi of CerS2 null mice with a Cy3-coupled anti-ceramide antibody reveals accumulation of ceramide in bronchial and tracheal epithelial cells of CerS2 null mice. Representative images are shown. Data are means±s.d. of the fluorescence (in arbitrary units, a.u.). n=3. ***p<0.001.

FIG. 6. Summary of potency of various sphingoid long chain bases.

### DETAILED DESCRIPTION OF THE INVENTION

The present invention provides sphingoid long chain bases (LCB) and pharmaceutical compositions comprising same for use in preventing, ameliorating and treating bacterial infections, including but not limited to Pseudomonas aeruginosa (P. aeruginosa). In preferred embodiments, the sphingoid LCBs are non-natural, i.e., they do not occur in nature. Advantageously, non-natural sphingoid LCBs do not undergo metabolism within a cell, e.g., by ceramidase, thereby not interfering with ceramide de novo or recycling synthesis pathways.

As exemplified hereinbelow, it is now shown that human CF patients, mice lacking Cftr (CF mice) and mice lacking ceramide synthase 2 (CerS2 null mice) contain very low levels of sphingosine in bronchial and tracheal epithelial cells, while levels of ceramide are significantly increased. Surprisingly, CF and CerS2 null mice are highly susceptible to pulmonary infection with P. aeruginosa. The present invention is based, in part, on the unexpected discovery that direct inhalation of sphingosine or of an analog, FTY720, prevented pulmonary P. aeruginosa infection in CerS2 null and CF mice. Moreover, sphingosine, FTY720 as well as a variety of sphingoid LCBs, prevent bacterial growth in vitro.

As disclosed hereinbelow, it has now unexpectedly been found that accumulation of ceramide in lungs of ceramide synthase 2 (CerS2)-deficient mice as well as in cystic fibrosis (CF) mice greatly facilitates pulmonary infection, and the high infection susceptibility of these mice is corrected by inhalation with acid ceramidase, which reduces bronchial ceramide and generates sphingosine. Likewise, inhalation of sphingosine or its derivative, FTY720 (2-amino-2-[2-(4-octylphenyl)ethyl]propane-1,3-diol, also known as fingolimod), prevents pulmonary P. aeruginosa infection of CerS2-deficient or CF mice. Without wishing to be bound by any particular theory or mechanism of action, the data presented herein establishes novel paradigms, namely that (i) mammalian sphingolipids (SLs), in particular ceramide and sphingosine, directly act on P. aeruginosa and determine its virulence and (ii) alterations in lung ceramide/sphingosine ratios influence the course of P. aeruginosa infection.

Compounds As contemplated herein, compounds that are useful in treating bacterial infections, can be represented by the structure of formula (I) or (II), with each possibility representing a separate embodiment of the present invention.

In one embodiment, the compound is represented by the structure of formula (I).

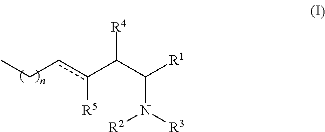

wherein
R¹ is H, CH₃, or —CH₂OR^{a} wherein R^{a} is H, C1-C4 alkyl or a sugar moiety;
R² and R³ are each selected from H or C1-C4 alkyl;
R⁴ and R⁵ are each independently H or OH;
n is an integer between 10 and 16; and
[symbol] is an optional double bond,
with the proviso that the following compounds are excluded:
   D-erythro-sphingosine;
   4-D-hydroxysphinganine (phytosphingosine);
   D-erythro-dihydrosphingosine (D-erythro-sphinganine);
   D,L-erythro-dihydrosphingosine (D,L-erythro-sphinganine); and
   D,L-threo-dihydrosphingosine (D,L-threo-sphinganine);
and salts, hydrates, solvates, polymorphs, optical isomers, enantiomers, diastereomers, epimers, and mixtures thereof.

In another embodiment, the compound is represented by the structure of formula (II).

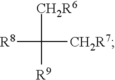

wherein
one of R⁶, R⁷ and R⁸ is NR^{b}R^{c}, and the other two are OR^{d}, wherein R^{b}, R^{c} and R^{d} are each H or C1-C4 alkyl;
R⁹ is a alkyl-phenyl wherein the phenyl is substituted by a C6-C20 alkyl group; and salts, hydrates, solvates, polymorphs, optical isomers, enantiomers, diastereomers, epimers, and mixtures thereof.

In addition, when the bacterial infection is associated with a lung disorder or disease (e.g., CF, COPD), the compound may also represented by the structure of formula (I′).

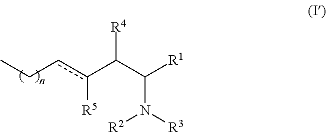

wherein
R¹ is H, CH₃, or —CH₂OR^{a} wherein R^{a} is H, C1-C4 alkyl or a sugar moiety;
R² and R³ are each selected from H or C1-C4 alkyl;
R⁴ and R⁵ are each independently H or OH;
n is an integer between 10 and 16; and
[symbol] is an optional double bond,
with the proviso that the following compounds are excluded:
   D-erythro-sphingosine;
   D-erythro-dihydrosphingosine (D-erythro-sphinganine);
and salts, hydrates, solvates, polymorphs, optical isomers, enantiomers, diastereomers, epimers, and mixtures thereof.

In one embodiment, the compound is represented by the structure of formula (I) or (I′). In some embodiments, the compound is a non-natural sphingoid LCB. In particular embodiments, the compound is selected from non-natural isomers of sphingosine, dihydrosphingosine and phytosphingosine. Each possibility represents a separate embodiment of the present invention.

In another embodiment, the compound is positively charged in vivo. In yet another embodiment, the compound is not a substrate of ceramide synthase.

In specific embodiments, the compound is selected from a D-threo, L-threo and L-erythro isomer of the compound of formula (I) or (I′). In another embodiment, the sphingoid LCB comprises at least 18 carbons, at least 19 carbons, preferably at least 20 carbons. Each possibility represents a separate embodiment of the present invention.

In another embodiment, the compound is represented by the structure of formula (I), wherein R¹ is CH₂OH. In another embodiment, R¹ is selected from H, CH₃, CH₂OCH₃, —CH₂—O-galactosyl and —CH₂—O-glucosyl.

In some embodiments, R^{a} represents a sugar moiety. The sugar may be a monosaccharide selected from a 5 carbon sugar (pentose) and 6 carbon sugar (hexose). Preferably the sugar is in ring form (furanose or pyranose). It is preferred that the present monosaccharide derivatives are derived from pentoses with the general formula C₅H₁₀O₅ or are from hexoses with the general formula C₆H₁₂O₆. Suitable pentoses may be selected from the group consisting of arabinose, ribose, and xylose. Suitable hexoses may be selected from the group consisting of allolose, altriose, fructose, galactose, glucose, gulose, inositol, mannose, and sorbose. Preferably, the novel monosaccharide derivatives are derived from inositol, galactose or glucose. Each possibility represents a separate embodiment of the present invention.

In another embodiment, the compound is represented by the structure of formula (I), wherein R² and R³ are each H. In another embodiment, the compound is represented by the structure of formula (I), wherein R⁴ is OH and R⁵ is H.

In another embodiment, the compound is represented by the structure of formula III

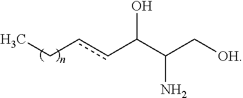

In some embodiments, the compound is represented by the structure of formula (I), wherein [symbol] is a double bond. In other embodiments [symbol] is a single bond.

In additional embodiments, the compound is represented by the structure of formula (II). In particular embodiments, one of R⁶, R⁷ and R⁸ is NH₂, and the other two are OH. In another embodiment, the compound of formula (II) is represented by the structure of formula IIa or IIb:

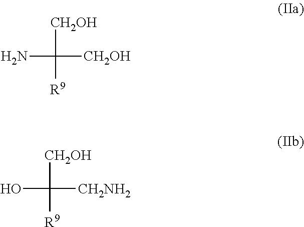

In particular embodiments, R⁹ is an alkyl-phenyl, wherein the phenyl is substituted by a C6-C10 alkyl, preferably by a C8 alkyl. In another particular embodiment, R⁹ is represented by the structure:

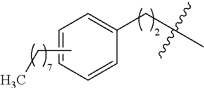

According to some embodiments, the LCB compound is a D-erythro isomer. According to additional embodiments, the LCB compound is a D-threo isomer. According to further embodiments, the LCB compound is a L-erythro isomer. According to yet additional embodiments, the LCB compound is a L-threo isomer.

According to particular embodiments, the LCB the compound is selected from the group consisting of: D-erythro-C20-sphingosine; D-threo-sphingosine; L-threo-sphingosine; L-erythro-sphingosine; D-threo-dihydrosphingosine (D-threo-sphinganine); L-threo-dihydrosphingosine (L-threo-sphinganine); L-erythro-dihydro sphingosine (L-erythro-sphinganine); 3-deoxy-D-erythro-sphingosine; 1-deoxy-D-erythro-dihydrosphingosine; 1-desoxymethylsphingosine; 1-deoxy-D-erythro-sphingosine; D-erythro-C16-sphingosine; Monomethyl D-erythro-sphingosine; Galactosyl D-erythro-sphingosine (psychosine); Glucosyl sphingosine; 2-amino-2-[2-(4-octylphenyl)ethyl]-1,3-propanediol (Fingolimod, FTY720); 2-(aminomethyl)-4-(4-octylphenyl)butane-1,2-diol; and 2-amino-2-(methoxymethyl)-4-(4-octylphenyl)butan-1-ol. In addition, when the compound is represented by the structure of formula (I′), the following additional compounds are included: 4-D-hydroxysphinganine (phytosphingosine); D-erythro-dihydrosphingosine (D-erythro-sphinganine); L-erythro-dihydro sphingosine (L-erythro-sphinganine); D-threo-dihydrosphingosine (L-threo-sphinganine); and L-threo-dihydrosphingosine (L-threo-sphinganine). Each possibility represents a separate embodiment of the present invention.

The term “C1-C4 alkyl”, used herein alone or as part of another group denotes linear and branched, saturated groups having from 1 to 4 carbon atoms, for example methyl, ethyl, propyl, isopropyl, butyl, sec-butyl or -butyl.

All stereoisomers of the above compounds are contemplated, either in admixture or in pure or substantially pure form. The compounds of the present invention can have asymmetric centers at any of the atoms. Consequently, the compounds can exist in enantiomeric or diastereomeric forms or in mixtures thereof. The present invention contemplates the use of any racemates (i.e. mixtures containing equal amounts of each enantiomers), enantiomerically enriched mixtures (i.e., mixtures enriched for one enantiomer), pure enantiomers or diastereomers, or any mixtures thereof. The chiral centers can be designated as R or S or R,S or d,D, l,L or d,l, D,L. The sugar residues include residues of D-sugars, L-sugars, erythro isomers, threo isomers, D-erythro, L-erythro, D-threo, L-threo, or racemic derivatives of sugars, or any combination thereof.

One or more of the compounds of the invention, may be present as a salt. The term “salt” encompasses both basic and acid addition salts, including but not limited to salts with amine nitrogens, and include salts formed with the organic and inorganic anions discussed below. Furthermore, the term includes salts that form by standard acid-base reactions with basic groups (such as amino groups) and organic or inorganic acids. Such acids include, but are not limited to, hydrochloric, hydrofluoric, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicylic, methanesulfonic, benzenesulfonic, sorbic, picric, benzoic, cinnamic, and like acids.

The present invention also includes solvates of the compounds of the present invention and salts thereof. “Solvate” means a physical association of a compound of the invention with one or more solvent molecules. This physical association involves varying degrees of ionic and covalent bonding, including hydrogen bonding. In certain instances the solvate will be capable of isolation. “Solvate” encompasses both solution-phase and isolatable solvates. Non-limiting examples of suitable solvates include ethanolates, methanolates and the like. “Hydrate” is a solvate wherein the solvent molecule is water.

The present invention also includes polymorphs of the compounds of the present invention and salts thereof. The term “polymorph” refers to a particular crystalline state of a substance, which can be characterized by particular physical properties such as X-ray diffraction, IR spectra, melting point, and the like.

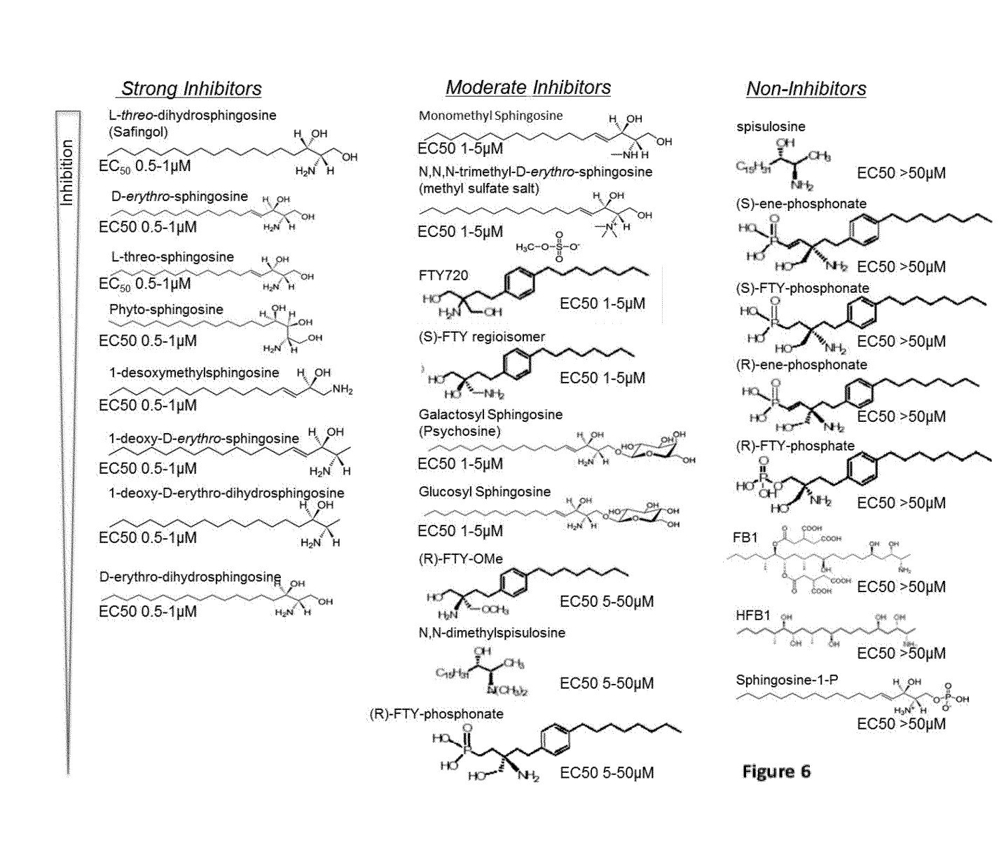

Without wishing to be bound by any particular theory or mechanism of action, and as further demonstrated in the experimental section below (see, Table 1 and FIG. 6), some preferred structural features for inhibition of bacterial growth by sphingoid long chain bases and their analogs include:
1) As contemplated herein, the sphingoid LCBs of the present invention have been categorized by their ability to act as bacterial inhibitors, as follows: strong inhibitors (EC50 of 0.5-1 μM), moderate inhibitors (EC50 of 1-5 μM) and non-inhibitors (EC50 >50 μM). The latter do not have any inhibitory effects on bacterial growth up to 50 μM.
2) The preferred features for strong inhibition appear to require a:
   a) Sphinganine or sphingosine long chain base (where the difference between sphingosine and sphinganine (dihydrosphingosine) is a double bond at the 4,5 position), with the D-erythro enantiomers of each both inhibiting in a similar fashion. Thus, the double bond at position 4,5 does not seem to play a crucial role.
   b) The L-threo-dihydrosphingosine enantiomer inhibits as well as the natural occurring D-erythro enantiomer as does the L-threo enantiomer. Thus, it appears that there is no stereospecificity requirement to the long chain base backbone. However, the non-natural LCBs such as but not limited to the L-threo analogs may be better for use in combatting bacteria since they are metabolized less in mammals than the natural D-erythro analogs.
   c) Phytosphingosine is also a strong inhibitor. Thus, an additional OH moiety may be added at C-3 of the long chain base
   d) 1-desoxylmethyl sphingosine, which cannot be metabolized to higher order sphingolipids, and 1-deoxy-sphingosine/dihydrosphingosine, are also strong inhibitors. Thus, further metabolism of the long chain bases is not required for combating bacteria.
   e) Inhibition of bacterial growth is positively influenced with increasing the chain length of the LCB. C20-sphingosine was found to be more potent than C18-sphingosine suggesting that longer LCBs might be even more potent inhibitors. No inhibition occurs with LCBs containing less than 16 carbons.
   f) Since bacteria are positively charged, it is likely that increasing the positive charge of the compound will be beneficial. Thus, a preferred LCB is one that is positively charged in vivo. In this respect, modification of the amine at position 2 (e.g., N-acetyl-dihydrosphingosine) reduces the activity, which may be due to the reduction of the overall positive charge of the molecule introduced by this modification.
   g) A preferred LCB has unmodified hydroxy and amino groups at C1-C3 (e.g., OH at C1 and C3, and NH₂ at C2).
3) Moderate, but still potent inhibition is given by compounds with the following characteristics:
   a) FTY720 analogs, which contain a modified long chain base.
   b) The presence of a head group at the C1 position somewhat reduces efficacy in bacterial killing (i.e. glucosyl sphingosine), however such compounds are still inhibitors at low micromolar range.
4) The following structural features prevent bacterial killing
   a) the presence of a phosphate at the C1 position on either the natural sphingoid long chain base or on FTY.
   b) A negatively charged compounds which for example contain phosphate group.

In some embodiments, some structural features required for bacterial killing include a sphingoid long chain base in either the natural (D-erythro) or non-natural (L-threo) configuration and the lack of a head group in the C1 position (i.e. phosphate). In some embodiments, long chain bases in the L-threo, L-erythro or D-threo conformation, including or lacking an OH substituent on C-1 that are optionally strongly positively charged, are preferred as agents used for bacterial killing with the least side-effects (see FIG. 6).

The term “non-natural” or “unnatural”, used herein interchangeably, denotes a sphingoid LCB that does not occur in nature, i.e., in biological systems, preferably mammals. The term does not include compounds that are present in biological systems (e.g., mammalian cells) as part of or as a result of biosynthetic pathways. Examples of naturally occurring sphingoid LCBs include, but are not limited to D-erythro derivatives of sphingosine, dihydrosphingosine (sphinganine), and phytosphingosine. Isomers of such compounds other than the D-erythro (i.e., D-threo, L-threo and L-erythro) do not occur in nature, and are therefore considered non-natural sphingoid LCBs. A list of natural LCBs are disclosed in Pruett et al. J Lipid Res. 2008; 49(8): 1621-1639, the contents of which are incorporated by reference in its entirety.

### Therapeutic Uses

As contemplated herein, the present invention is based on the finding that sphingoid LCBs of formula (I) or (II), are active as antibacterial agents, especially against bacteria associated with lung infections (e.g., in COPD or CF patients).

Thus, in one, the present invention relates to a method for preventing or treating a bacterial infection in a subject in need thereof comprising administering to the subject, by inhalation, a therapeutically effective amount of a pharmaceutical composition comprising a sphingoid long chain base (LCB) and a pharmaceutically acceptable carrier, thereby preventing or treating a bacterial infection, wherein the sphingoid LCB is represented by formula (I) or (II). When the bacterial infection is associated with a lung disorder, disease or infection, the compound may also be represented by the structure of formula (I′). Each possibility represents a separate embodiment of the present invention.

In another aspect the present invention relates to a pharmaceutical composition comprising as an active ingredient a sphingoid long chain base (LCB) compound and a pharmaceutically acceptable carrier, for use in preventing or treating a bacterial infection, wherein the pharmaceutical composition is formulated for inhalation, and wherein the sphingoid LCB compound is represented by the structure of formula (I) or formula (II). When the bacterial infection is associated with a lung disorder, disease or infection, the compound may also be represented by the structure of formula (I′). Each possibility represents a separate embodiment of the present invention.

In another aspect, the present invention relates to the use of a pharmaceutical composition comprising a sphingoid long chain base (LCB) compound and a pharmaceutically acceptable carrier, wherein the composition is formulated for inhalation, for the manufacture of a medicament for the prevention or treatment of a bacterial infection, wherein the pharmaceutical composition is formulated for inhalation, and wherein the sphingoid LCB compound is represented by the structure of formula (I) or formula (II). When the bacterial infection is associated with a lung disorder, disease or infection, the compound may also be represented by the structure of formula (I′). Each possibility represents a separate embodiment of the present invention.

Underlying disease conditions can predispose a subject to acute and/or chronic bacterial infections. As used herein a “disease condition” refers to a pathological disease or condition of any kind or origin, which a subject harbors. Accordingly, disease conditions include the subject matter identified by the following diseases and/or terms including, but not limited to, e.g., a respiratory disease, lung disease, Cystic Fibrosis (“CF”), chronic obstructive pulmonary disease (“COPD”), emphysema, asthma, pulmonary fibrosis, chronic bronchitis, pneumonia, pulmonary hypertension, lung cancer, sarcoidosis, necrotizing pneumonia, asbestosis, aspergilloma, aspergillosis, acute invasive atelectasis, eosinophilic pneumonia, pleural effusion, pneumoconiosis, pneumocystosis, pneumothorax, pulmonary actinomycosis, pulmonary alveolar proteinosis, pulmonary anthracis, pulmonary arteriovenous malformation, pulmonary edema, pulmonary embolus, pulmonary histiocytosis X (eosinophilic granuloma), pulmonary nocardiosis, pulmonary tuberculosis, pulmonary veno-occlusive disease and/or rheumatoid lung disease. Such diseases typically manifest an increased susceptibility of a subject for bacterial infection, i.e., compared to subjects not afflicted with a disease condition. For example, subjects suffering from CF or COPD, may possess a high susceptibility for acquiring acute and/or chronic bacterial infections.

Examples of bacterial strain that can be treated by the compositions of the invention are all Gram-negative and Gram-positive bacteria and in particular gram negative and gram positive bacteria. The compounds of the present invention are effective against a wide variety of Gram-positive and Gram-negative bacteria, for example Staphylococcus species (e.g., Staphylococcus aureus), Acinetobacter species (e.g., baumanii Acinetobacter), Pseudomonas species (e.g., Pseudomonas aeruginosa), Burkholderia species, and Mycobacteria species (typical and atypical), and others. In some currently preferred embodiments, the bacterial infection is a Pseudomonas infection. In other current preferred embodiments, the Pseudomonas infection is a Pseudomonas aeruginosa infection. Additional non-limiting examples of bacteria include:

Additional bacterial strains treatable by the compounds of the present invention include, but are not limited to Acinetobacter baumanii, Actinobacillus species, Actinomycetes, Actinomyces species (such as Actinomyces israelii and Actinomyces naeslundii), Aeromonas species (such as Aeromonas hydrophila, Aeromonas veronii biovar sobria (Aeromonas sobria), and Aeromonas caviae), Anaplasma phagocytophilum, Alcaligenes xylosoxidans, Acinetobacter baumanii, Actinobacillus actinomycetemcomitans, Bacillus species (such as Bacillus anthracis, Bacillus cereus, Bacillus subtilis, Bacillus thuringiensis, and Bacillus stearothermophilus), Bacteroides species (such as Bacteroides fragilis), Bartonella species (such as Bartonella bacilliformis and Bartonella henselae, Bifidobacterium species, Bordetella species (such as Bordetella pertussis, Bordetella parapertussis, and Bordetella bronchiseptica), Borrelia species (such as Borrelia recurrentis, and Borrelia burgdorferi), Brucella species (such as Brucella abortus, Brucella canis, Brucella melintensis and Brucella suis), Burkholderia species (such as Burkholderia pseudomallei and Burkholderia cepacia), Campylobacter species (such as Campylobacter jejuni, Campylobacter coli, Campylobacter lari and Campylobacter fetus), Capnocytophaga species, Cardiobacterium hominis, Chlamydia trachomatis, Chlamydophila Pneumococcus species, pneumoniae, Chlamydophila psittaci, Citrobacter species Coxiella burnetii, Corynebacterium species (such as, Corynebacterium diphtheriae, Corynebacterium jeikeum Corynebacterium, and Corynebacterium dipththeriae), Clostridium species (such as Clostridium perfringens, Clostridium difficile, Clostridium botulinum and Clostridium tetani), Eikenella corrodens, Enterobacter species (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus species (such as Enterococcus faecalis and Enterococcus faecium) Ehrlichia species (such as Ehrlichia chafeensia and Ehrlichia canis), Erysipelothrix rhusiopathiae, Eubacterium species, Francisella tularensis, Fusobacterium nucleatum, Gardnerella vaginalis, Gemella morbillorum, Haemophilus species (such as Haemophilus influenzae, e.g., type B or non-typable, Haemophilus ducreyi, Haemophilus aegyptius, Haemophilus parainfluenzae, Haemophilus haemolyticus and Haemophilus parahaemolyticus, Helicobacter species (such as Helicobacter pylori, Helicobacter cinaedi and Helicobacter fennelliae), Kingella kingii, Klebsiella species (such as Klebsiella pneumoniae, Klebsiella granulomatis and Klebsiella oxytoca), Lactobacillus species, Listeria monocytogenes, Leptospira interrogans, Legionella pneumophila, Leptospira interrogans, Peptostreptococcus species, Moraxella catarrhalis, Morganella species, Mobiluncus species, Micrococcus species, Mycobacterium species (such as Mycobacterium leprae, Mycobacterium tuberculosis, Mycobacterium intracellulare, Mycobacterium avium, Mycobacterium bovis, and Mycobacterium marinum), Mycoplasm species (such as Mycoplasma pneumoniae, Mycoplasma hominis, and Mycoplasma genitalium), Nocardia species (such as Nocardia asteroides, Nocardia cyriacigeorgica and Nocardia brasiliensis), Neisseria species (such as Neisseria gonorrhoeae and Neisseria meningitidis), Pasteurella multocida, Plesiomonas shigelloides. Prevotella species, Porphyromonas species, Prevotella melaninogenica, Proteus species (such as Proteus vulgaris and Proteus mirabilis), Providencia species (such as Providencia alcalifaciens, Providencia rettgeri and Providencia stuartii), Pseudomonas aeruginosa, Propionibacterium acnes, Rhodococcus equi, Rickettsia species (such as Rickettsia rickettsii, Rickettsia akari and Rickettsia prowazekii, Orientia tsutsugamushi (formerly: Rickettsia tsutsugamushi) and Rickettsia typhi), Rhodococcus species, Serratia marcescens, Stenotrophomonas maltophilia, Salmonella species (such as Salmonella enterica, Salmonella typhi, Salmonella paratyphi, Salmonella enteritidis, Salmonella cholerasuis and Salmonella typhimurium), Serratia species (such as Serratia marcesans and Serratia liquifaciens), Shigella species (such as Shigella dysenteriae, Shigella flexneri, Shigella boydii and Shigella sonnei), Staphylococcus species (such as Staphylococcus aureus, Staphylococcus epidermidis, Staphylococcus hemolyticus, Staphylococcus saprophyticus), Streptococcus species (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema species (such as Treponema carateum, Treponema petenue, Treponema pallidum and Treponema endemicum, Tropheryma whippelii, Ureaplasma urealyticum, Veillonella species, Vibrio species (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia species (such as Yersinia enterocolitica, Yersinia pestis, and Yersinia pseudotuberculosis) and Xanthomonas maltophilia among others.

The term “lung disorder” or “lung disease”, used herein interchangeably, refers to conditions that affect the lung, and are inclusive of lung infections that are associated with lung conditions or diseases. Such conditions include, but are not limited to, cystic fibrosis (CF), chronic obstructive pulmonary disease (COPD)

The methods of the invention both ex-vivo and in the body of the subject may further comprise co administration of at least one additional anti-bacterial agent such as state of the art antibiotics.

The term “anti-bacterial” may refer to one or more of the following effects: killing the bacteria (bacteriocide), causing halt of growth of bacteria (bacteriostatic), prevention of bacterial infection, and decrease in bacterial virulence.

The term “combating bacteria” or “treating bacterial infection” may refer to one of the following: decrease in the number of bacteria, killing or eliminating the bacteria, inhibition of bacterial growth (stasis), inhibition of bacterial infestation, decrease in bacterial virulence, or any combination thereof.

As used herein, the term “administering” refers to bringing in contact with a compound of the present invention. Administration can be accomplished to cells or tissue cultures, or to living organisms, for example humans. In one embodiment, the present invention encompasses administering the compounds of the present invention to a human subject.

A “therapeutic” treatment is a treatment administered to a subject who exhibits signs of pathology for the purpose of diminishing or eliminating those signs. A “therapeutically effective amount” is that amount of compound which is sufficient to provide a beneficial effect to the subject to which the compound is administered.

“Preventing” and “prevent” means avoiding the onset of a clinically evident disease progression altogether or slowing the onset of a pre-clinically evident stage of a disease in individuals at risk. Prevention includes prophylactic treatment of those at risk of developing a disease.

### Pharmaceutical Compositions

The present invention provides, in some embodiments, pharmaceutical compositions comprising as an active ingredient a sphingoid long chain base (LCB) and a pharmaceutically acceptable carrier. The term “pharmaceutically acceptable carrier” refers to a vehicle which delivers the active components to the intended target and which does not cause harm to humans or other recipient organisms. As used herein, “pharmaceutical” will be understood to encompass both human and animal pharmaceuticals. Useful carriers include, for example, water, acetone, ethanol, ethylene glycol, propylene glycol, butane-1,3-diol, isopropyl myristate, isopropyl palmitate, or mineral oil. Use of detergents such as n-Octyl-β-D-Glucopyranoside (OGP) is also contemplated. Methodology and components for formulation of pharmaceutical compositions are well known, and can be found, for example, in Remington's Pharmaceutical Sciences, Eighteenth Edition, A. R. Gennaro, Ed., Mack Publishing Co. Easton Pa., 1990, the contents of which are incorporated by reference in their entirety.

Typically, pharmaceutical composition are formulated in any form appropriate to the mode of administration, for example, solutions, colloidal dispersions, emulsions (oil-in-water or water-in-oil), suspensions, creams, lotions, gels, foams, sprays, aerosol, ointment, tablets, suppositories, and the like. In exemplified embodiments, the pharmaceutical compositions of the present invention are formulated for aerosol administration for inhalation by a subject in need thereof.

A therapeutically effective amount of a pharmaceutical composition of the invention is an amount that when administered to a patient is capable of preventing or ameliorating infection, e.g., bacterial infection. The effective amount of an agent or composition of the present invention administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions of the present invention can also be administered in combination with one or more additional therapeutic compounds. Precise amounts of active ingredient required to be administered depend on the judgment of the practitioner and are peculiar to each individual.

Typically, the therapeutic agent will be administered as a pharmaceutical formulation that includes the therapeutic agent and any pharmaceutically acceptable adjuvants, carriers, excipients, and/or stabilizers, and can be in solid or liquid form, such as tablets, capsules, powders, solutions, suspensions, or emulsions. The compositions preferably contain from about 0.01 to about 99 weight percent, more preferably from about 2 to about 60 weight percent, of therapeutic agent together with the adjuvants, carriers and/or excipients. In some embodiments, an effective amount ranges from about 0.001 mg/kg to about 500 mg/kg body weight of the subject. In some embodiments, the effective amount of the agent ranges from about 0.05 mg/kg to about 30 mg/kg, from about 0.1 mg/kg to about 30 mg/kg, from about 1 mg/kg to about 25 mg/kg, 30 from about 1 mg/kg to about 20 mg/kg, or from about 1 or 2 mg/kg to about 15 mg/kg.

In preferred embodiments, the composition of the invention is administered by intranasal or intraoral administration, using appropriate solutions, such as nasal solutions or sprays, aerosols or inhalants. Nasal solutions are usually aqueous solutions designed to be administered to the nasal passages in drops or sprays. Typically, nasal solutions are prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal and oral preparations for inhalation, aerosols and sprays are known and include, for example, antibiotics and antihistamines and are used for asthma prophylaxis.

For intranasal or intraoral administration the composition of the invention is provided in a solution suitable for expelling the pharmaceutical dose in the form of a spray, wherein a therapeutic quantity of the pharmaceutical composition is contained within a reservoir of an apparatus for nasal or intraoral administration. The apparatus may comprise a pump spray device in which the means for expelling a dose comprises a metering pump. Alternatively, the apparatus comprises a pressurized spray device, in which the means for expelling a dose comprises a metering valve and the pharmaceutical composition further comprises a conventional propellant. Suitable propellants include one or mixture of chlorofluorocarbons, such as dichlorodifluoromethane, trichlorofluoromethane, dichloro-tetrafluoroethane, hydrofluorocarbons, such as 1,1,1,2-tetrafluoroethane (HFC-134a) and 1,1,1,2,3,3,3-heptafluoropropane (HFC-227) or carbon dioxide. Suitable pressurized spray devices are well known in the art and include those disclosed in, inter alia, WO 92/11190, U.S. Pat. No. 4,819,834, U.S. Pat. No. 4,407,481 and WO 97/09034, when adapted for producing a nasal spray, rather than an aerosol for inhalation, or a sublingual spray. The contents of the aforementioned publications are incorporated by reference herein in their entirety. Suitable nasal pump spray devices include the VP50, VP70 and VP100 models available from Valois S. A. in Marly Le Roi, France and the 50, 70 and 100 ul nasal pump sprays available from Pfeiffer GmbH in Radolfzell, Germany, although other models and sizes can be employed. In the aforementioned embodiments, a pharmaceutical dose or dose unit in accordance with the invention can be present within the metering chamber of the metering pump or valve.

Methods of treating a disease according to the invention may include administration of the pharmaceutical compositions of the present invention as a single active agent, or in combination with additional methods of treatment. In some embodiments, the one or more additional agents are selected from the group consisting of one or more ceramide reducing agents (including but not limited to ceramidase or acid ceramidase), one or more acid sphingomyelinase inhibitors, one or more agents to reduce infection, and combinations thereof. Suitable agents to reduce infection include antibiotics (e.g., inhaled Tobramycin, TOBI), reagents that block binding of pathogens to lung epithelium, reagents to reduce mucus viscosity (e.g., Dornase alfa, Pulmozyme), chaperone reagents to enhance missing protein function (e.g., Ivacaftor, Kalydeco), and combinations thereof.

The methods of treatment of the invention may be in parallel to, prior to, or following additional methods of treatment.

The following examples are presented in order to more fully illustrate some embodiments of the invention. They should, in no way be construed, however, as limiting the broad scope of the invention.

### EXAMPLES

### Materials and Methods

Sphingosine levels were determined in the trachea and lung of wildtype (wt), Cftr- and CerS2 null mice (Teichgräber, V. et al. Nat Med 2008, 14, 382-391; Pewzner-Jung et al. J Biol Chem. 2010 Apr. 2; 285(14):10902-10) and human nasal tissues by (i) confocal microscopy analysis of paraffin sections stained with Cy3-coupled anti-sphingosine antibodies and (ii) by newly developed biochemical assays that employed sphingosine kinase assays to determine sphingosine in freshly isolated tracheal epithelial cells and on the surface of tracheal epithelial cells in situ. In the latter experiments the intact trachea was carefully opened and incubated with sphingosine kinase in the presence of [³²]PγATP. Lipids were extracted and the concentration of sphingosine-1-[³²]P was determined. In addition, surface sphingosine was immunoprecipitated from the surface of tracheal epithelial cells by binding of anti-sphingosine antibodies to the intact inner surface of the trachea, followed by washing, lysis, immobilization of immune complexes, extraction of bound sphingosine and quantification of sphingosine by kinase assays.

Pulmonary infection of CF or CerS2 mice was achieved by intranasal application of P. aeruginosa strains 762, ATCC 27853 or PA14. Mice inhaled acid ceramidase, sphingosine, FTY720 or a combination of sphingosine and ceramide 1 hr before or after pulmonary infection. The number of bacteria in the lung was determined by culturing aliquots of lysed lungs on agar plates. Clinical symptoms of the infection were scored.

The in vitro effect of sphingosine in the presence or absence of ceramide was determined by incubation of early-logarithmic grown P. aeruginosa with sphingosine or ceramide or both in different ratios and measurement of surviving bacteria measuring the number of colony forming units 24 and 48 hrs after treatment.

All results are shown as mean±S.D. p values were calculated by Student's t-test and significance is presented as a p value of either *=p<0.05, **=p<0.01, ***=p<0.001.

### Mice

CerS2 null and CF mice were generated as previously described (Teichgräber, 2008 ibid. and Pewzner-Jung 2010 ibid.). Two different CF mouse strains were used, cftr^{tm1Unc}-Tg^{(FABPCFTR)} (abbreviated Cftr^{KO}) Jaw mice that lack Cftr, but express human CFTR in the gut under control of a fatty acid binding protein (FABP) promoter. These mice were originally obtained from Jackson Laboratories (Bar Harbor, Me., USA) and backcrossed for 10 generations onto the C57BL/6 background. All data displayed are from Cftr^{KO} mice. To confirm data obtained with strain we employed a 2^{nd} strain, i.e. the B6.129P2(CF/3)-Cftr^{TgH(neoim)Hgu} (abbreviated Cftr^{MHH}) congenic mice that were established by strict brother-sister mating from the original Cftr^{TgH(neoim)Hgu} mutant mouse generated using insertional mutagenesis in the Cftr exon 10. The congenic Cftr^{MHH} strain was generated by backcrossing the targeted mutation into the B6 inbred background. The strain produces low levels of Cftr. Control mice were syngenic littermates: C57BL/6 for CF mice and C57BL/6; 129Sv/J F1 WT mice for CerS2 null mice. The inventors did not observe any significant differences between Cftr^{MHH} and Cftr^{KO} or between the two control mouse strains.

Mice were bred in a special pathogen free (SPF) facility at the Weizmann Institute of Science and the University of Duisburg-Essen animal facilities. Mice were handled according to protocols approved by the Weizmann Institute of Science and the University of Duisburg-Essen Animal Care Committee as per international guidelines.

### Mouse Inhalation

Inhalation was performed with a PARI Boy SX nebulizer (PARI GmbH, Starnberg, Germany). This nebulizer generates a fine aerosol mist by pumping the fluid with an air jet. The mice inhaled the aerosol via a mask that is part of an oral inhalation device for children (LL-Nebulizer); the mask was clipped at the sides to cover only the mouse's nose and the surrounding part of the face. Mice were inhaled with 800 μl of 0.9% NaCl or Ringer solution lacking any Cl⁻ (142 mM sodium gluconate, 4 mM potassium gluconate, 2 mM calcium gluconate, 5 mM HEPES, pH 7.4 adjusted with CH₃COOH or NaOH) containing sphingosine (125 μM), C16-ceramide (125 μM), acid ceramidase (80 μg purified protein), FT720 (125 μM) or C16-ceramide plus sphingosine (each 125 μM or 250 μM C16-ceramide+125 μM sphingosine). Approximately 10% of the volume that is applied to the mice is inhaled by the mice.

### Mouse Infection

P. aeruginosa were grown for 14 to 14.5 h on fresh tryptic soy agar (TSA) plates (Becton Dickinson Biosciences, Germany). Bacteria were then transferred into 40 mL of pre-warmed, sterile tryptic soy broth (TSB) (Becton Dickinson Biosciences, Germany) in Erlenmeyer flasks. The OD was adjusted to 0.225, and the bacteria were grown for 60 min at 37° C. with shaking at 125 rpm to obtain bacteria in the early log phase. The bacteria were then centrifuged for 10 min at 1,600×g, the supernatant was carefully removed, and the bacteria were resuspended in prewarmed H/S (132 mM NaCl, 20 mM HEPES [pH 7.4], 5 mM KCl, 1 mM CaCl₂, 0.7 mM MgCl₂, 0.8 mM MgSO₄). The bacterial OD was determined, and the bacteria were diluted in prewarmed H/S to a concentration of 1×10⁸ CFU per 20 μL. The mice were anesthetized with ether for 10 to 15 sec, and bacteria were carefully injected into the nose with a 30-gauge 1 mL syringe. The needle was covered with a tightly fitting, smooth plastic tube so that nasal injuries were avoided.

P. aeruginosa Counts in Lung

Lungs were removed, homogenised and lysed for 10 min in 5 mg/ml saponin to release intracellular bacteria. The samples were centrifuged for 10 min at 1,600×g, washed once with sterile H/S solution and homogenates were cultured on Tryptic Soy Agar (TSA) plates (Becton Dickinson Biosciences, Germany). Bacterial counts (CFU) were counted after 18 h.

### Measurement of Ceramide and Sphingosine by In Situ Kinase Assays

Mice were sacrificed and the trachea immediately removed. The trachea were carefully opened, washed in 150 mM sodium-acetate (pH 7.4), placed on a 30° C. pre-warmed plastic plate and incubated with 0.01 units diacylglycerol (DAG) kinase (Biomol, Germany) for the ceramide kinase assay or 0.001 units sphingosine kinase (R&D Systems, Germany) in 4 μl of 150 mM sodium-acetate (pH 7.4) for the sphingosine kinase assay. 1 mM adenosine triphosphate (ATP) and 10 μCi [³²P]γATP were added at the inner surface of the trachea. The kinase reaction for ceramide was performed for 15 min at 30° C. and terminated by transfer of the trachea into CHCl₃:CH₃OH:1N HCl (100:100:1, v/v/v) followed by 170 μL buffered saline solution (135 mM NaCl, 1.5 mM CaCl₂, 0.5 mM MgCl₂, 5.6 mM glucose, 10 mM HEPES [pH 7.2]) and 30 μL of a 100 mM EDTA solution were added. The sphingosine kinase reaction was terminated by adding the trachea in 100 μl H₂O, following addition of 20 μl 1N HCl, 800 μl CHCl₃/CH₃OH/1N HCl (100:200:1, v/v/v), 240 μl CHCl₃ and 2 M KCl. Phases were separated, the lower phase was collected, dried, dissolved in 20 μL of CHCl₃:CH₃OH (1:1, v/v) and separated on Silica G60 thin-layer chromatography (TLC) plates using CHCl₃/acetone/CH₃OH/acetic acid/H₂O (50:20:15:10:5, v/v/v/v/v) for ceramide and CHCl₃/CH₃OH/acetic acid/H₂O (90:90:15:5, v/v/v/v) for sphingosine as developing solvents. The TLC plates were exposed to radiography films, the spots were removed from the plates, and the incorporation of [³²P] into ceramide was measured by liquid scintillation counting. Ceramide and sphingosine were determined by comparison with a standard curve of C16- to C24-ceramides or C18-sphingosine. To vary surface pH, the pH of the 150 mM sodium-acetate buffer was varied from 7.4 to 5.0.

### Measurement of Ceramide and Sphingosine in Extracts of Freshly Isolated Tracheal Epithelial Cells

Epithelial cells were removed from the trachea by carefully scraping the inner surface of the trachea. For ceramide measurements, cells were extracted in 200 μl H₂O and 600 μl CHCl₃:CH₃OH:1N HCl (100:100:1, v/v/v), the lower phase was dried, resuspended in 20 μL of a detergent solution (7.5% [w/v] n-octyl glucopyranoside, 5 mM cardiolipin in 1 mM diethylenetriaminepentaacetic acid [DTPA]), and 70 μl of 0.01 units diacylglycerol (DAG) kinase (Biomol, Germany) in 0.1 M imidazole/HCl (pH 6.6), 0.2 mM DTPA (pH 6.6), 70 mM NaCl, 17 mM MgCl₂, 1.4 mM ethylene glycol tetraacetic acid; 1 μM ATP and 10 μCi [³²P]γATP were added. For sphingosine measurements, cells were extracted in CHCl₃/CH₃OH/1N HCl (100:200:1, v/v/v), the lower phase was dried and resuspended in detergent solution as above. The kinase reaction was initiated by addition of 0.001 units sphingosine kinase in 50 mM HEPES (pH 7.4), 250 mM NaCl, 30 mM MgCl₂ 1 mM adenosine triphosphate (ATP) and 10 μCi [³²P]γATP. The samples were incubated for 30 min at 37° C. with 350 rpm shaking and processed as above.

### Kinase Assays of Sphingosine Immunoprecipitates

The luminal epithelial surface of the trachea was incubated with 50 ng of an anti-sphingosine antibody (clone NHSPH, Alfresa Pharma Corporation, Japan) pre-coupled to protein L-agarose beads (Santa Cruz Inc.) at 4° C. for 30 min in 150 mM sodium-acetate (pH 7.4)+1% fetal calf serum (FCS). Alternatively, we allowed the antibody for 20 min in 150 mM sodium-acetate (pH 7.4)+1% FCS to bind to the surface of the trachea at 4° C. The trachea was then washed 3 times in 150 mM sodium-acetate (pH 7.4) and L-agarose was added. Samples were incubation for additional 20 min, washed 6-times with ice-cold H/S, and lysed in 125 mM NaCl, 25 mM Tris HCl (pH 7.4), 10 mM EDTA, 10 mM sodiumpyrophosphate, 3% Nonidet P40 and 10 μg/ml aprotinin and leupeptin for 10 min on ice. The remaining, insoluble tissue was removed and the agarose beads washed 5 times in the lysis buffer. The beads were pelleted, the supernatants discarded and the beads extracted using 200 μl H₂O and 600 μl CHCl₃/CH₃OH/1N HCl (100:200:1, v/v/v). The lower organic phase was collected, dried, resuspended in 20 μL of a detergent solution (7.5% [w/v] n-octyl glucopyranoside, 5 mM cardiolipin in 1 mM diethylenetriaminepentaacetic acid [DTPA]), and sonicated for 10 min. The kinase reaction was started by the addition of 70 μL of a reaction mixture containing 2 μL sphingosine kinase in 50 mM HEPES (pH 7.4), 250 mM NaCl, 30 mM MgCl₂ 1 mM ATP and 10 μCi [³²P]γATP. The kinase reaction was terminated after 30 min and samples were further extracted and processed as above.

Treatment with Cytochalasin B

Trachea were incubated with 10 μM cytochalasin B for 20 min prior to kinase assays or sphingosine immunopreciptations. Surface sphingosine was measured by an in situ kinase assay or immunoprecipitation as above. As controls we incubated in parallel trachea with cytochalasin B, infected the trachea with 1×10⁶ CFU P. aeruginosa ATCC 27853 and determined uptake of the bacteria. The trachea was washed after infection, incubated for 60 min in 100 μg/ml polymixin, washed, lysed in 5 mg/ml saponin for 10 min, the samples were centrifuged for 10 min at 1,600×g the pellets resuspended in H/S, aliquots were plated on TSA and grown overnight to quantify the number of intracellular bacteria in the presence or absence of cytochalasin.

### Immunohistochemical Analysis of Ceramide, Sphingosine and Sphinganine

Animals were sacrificed, lungs or trachea were immediately removed, fixed for 36 h in 4% buffered PFA in PBS, embedded in paraffin and trimmed to 6 μm. The sections were dewaxed, incubated for 30 min with Pepsin Digest All, (Invitrogen Life Technologies, USA) at 37° C., washed, and blocked for 10 min with PBS, 0.05% Tween 20, and 5% fetal calf serum (FCS). They were then immunostained for 45 min with the anti-ceramide antibody clone S58-9 (Glycobiotech, Germany) or anti-sphingosine antibody (clone NHSPH, Alfresa Pharma Corporation, Japan). Anti-ceramide antibodies were diluted 1:100, anti-sphingosine antibodies 1:1000 in H/S+1% FCS, washed 3 times in PBS+0.05% Tween 20, incubated for 45 min with Cy3-coupled donkey anti-mouse IgM F(ab)₂ fragments (Jackson ImmunoResearch), washed again three times in PBS+0.05% Tween 20 and once in PBS, and embedded in Mowiol. Immunofluorescence was measured with the Leica TCS SL software program, version 2.61 (Leica, Germany). Fluoresence in the epithelial cell layer was quantified using 10-25 areas per sample measuring the fluorescence in the apical ⅓ of bronchial or tracheal epithelial cells. Quantification was performed using Photoshop software. To exclude binding of the anti-sphingosine antibody to sphinganine, the trachea from WT or CF mice were removed, incubated in vitro with 10 μM sphinganine in 150 mM sodium acetate (pH 7.4) for 30 min, washed 3 times in 150 mM sodium acetate (ph 7.4), fixed for 36 hrs in 2% PFA (pH 7.3) and stained as above with anti-sphingosine antibodies.

In Vitro Incubations of Bacteria with Sphingolipids

P. aeruginosa strain 762 or ATCC 27853 were grown in LB medium at 37° C. until the early logarithmic phase (OD₅₅₀ 0.2). Bacteria were then diluted in PBS to a concentration of 5×10³ CFU/ml. Stock solutions of sphingolipids (5 mM in 7.5% n-Octyl-β-D-Glucopyranoside (OGP) or in DMSO were prepared by sonication. Sphingolipids were added to the bacteria (0.1-50 μM in 0.075% OGP or 1% DMSO). Bacteria were incubated with sphingolipids for 2 hr at 37° C. and a sample of 200 μl bacteria solution were plated on LB agar plates, incubated overnight at 37° C. and colonies were counted.

### Sytox Green Staining

Bacteria at early logarithmic phase were incubated in the presence or absence of sphingosine or C16-ceramide (C16-cer) (50 μM in 1% DMSO) for 30 minutes at 37° C. Bacteria were then washed in PBS, centrifuged at 200×g, resuspended in 20 mM sodium phosphate buffer (pH 7.4), and incubated with 1 μM SYTOX green (Molecular Probes, Life Technologies, USA) for 10 min in the dark. Sample were examined under fluorescence microscope, and bacteria were counted both in bright field and under green fluorescent filter in two independent experiments.

### Example 1

### Sphingosine is Expressed on the Surface of Airway Epithelial Cells, but is Absent in CF Patients and in CF and CerS2 Null Mice

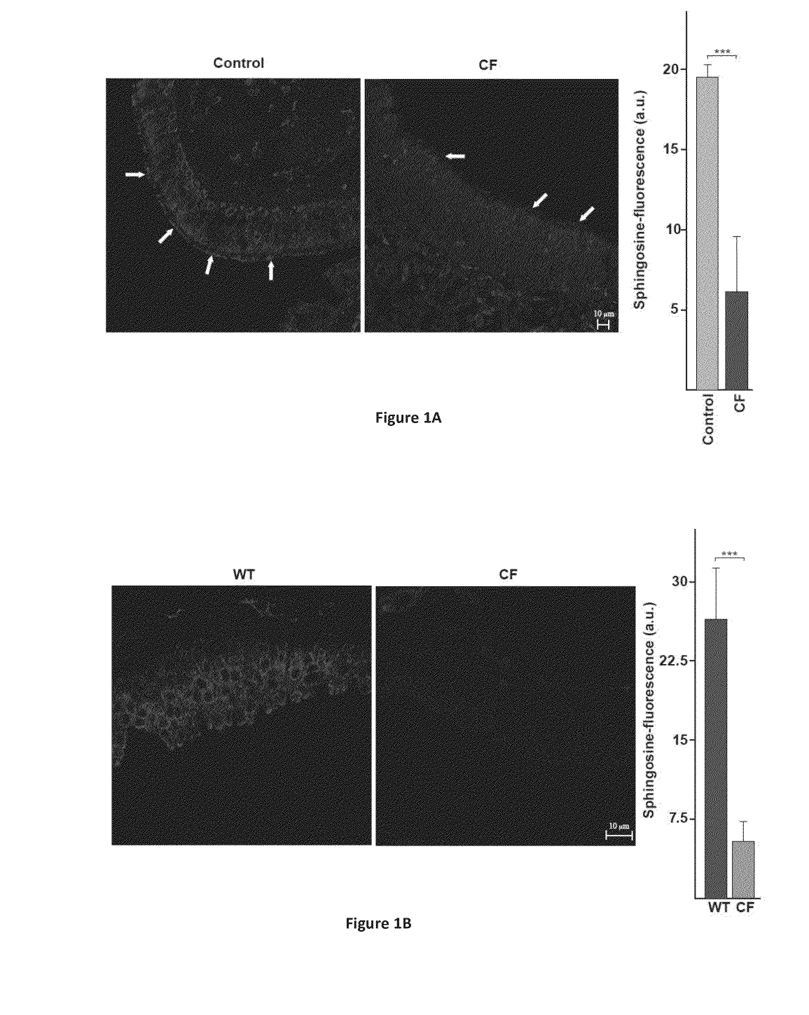

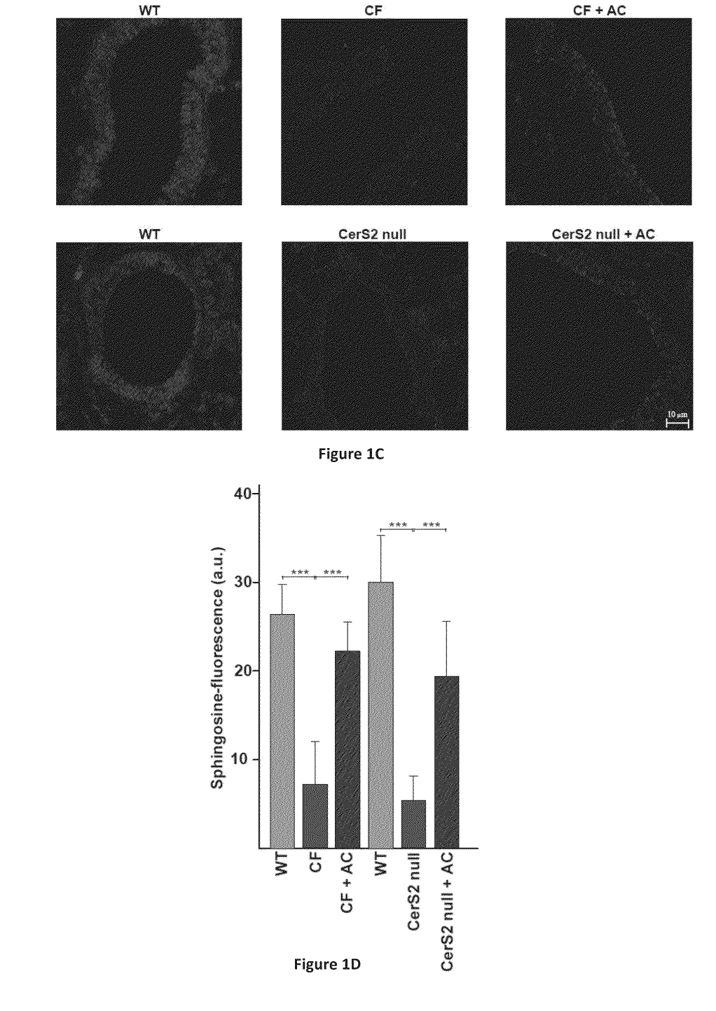

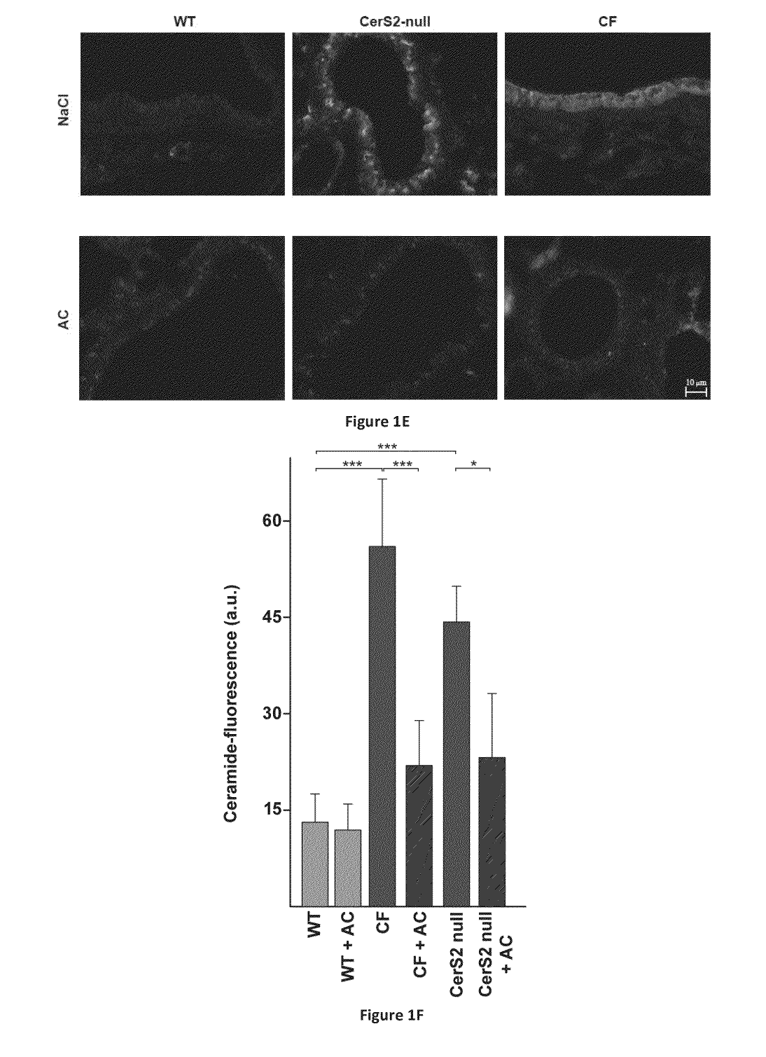

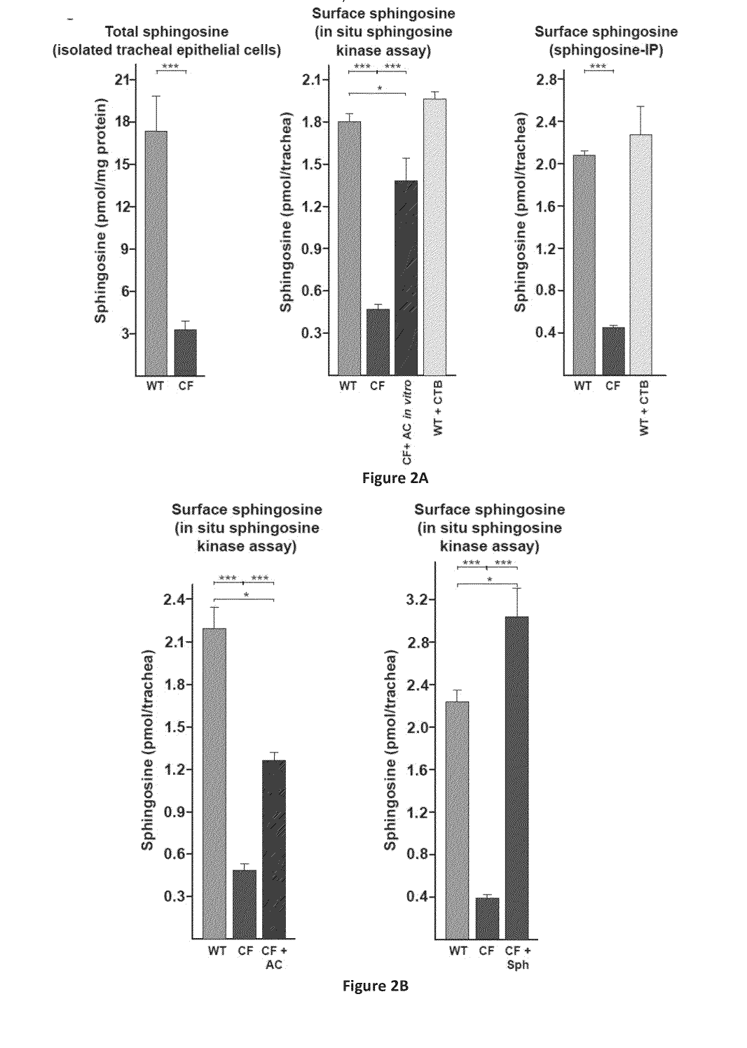

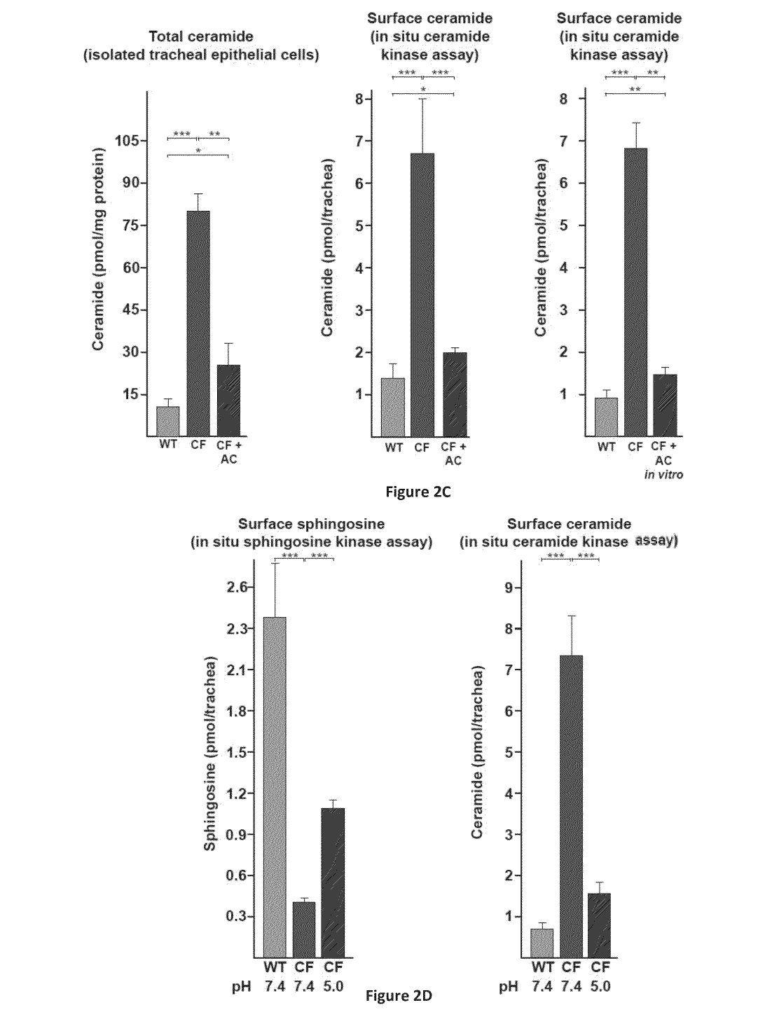

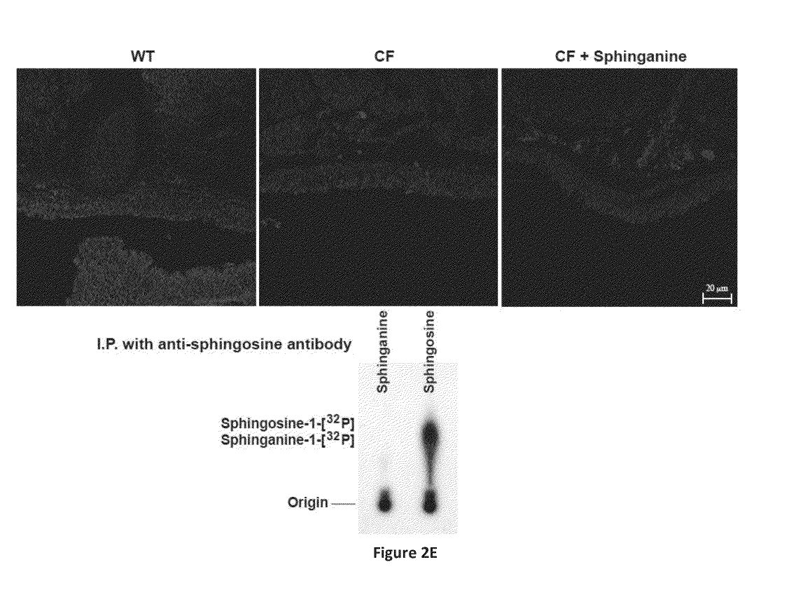

To examine whether sphingosine plays a role in CF, the inventors analyzed sphingosine levels in nasal epithelial cells obtained from individuals with CF or from healthy controls (FIG. 1A). Immunohistochemical analysis using an anti-sphingosine antibody revealed that sphingosine was abundantly expressed in human nasal epithelial cells obtained from healthy individuals (for validation of the specificity of the antibody, see FIG. 2E). Surprisingly, sphingosine levels were markedly reduced in human nasal epithelial cells from CF patients and almost undetectable on the surface of nasal epithelial cells of CF patients, the surface that is in direct contact with invading bacteria (FIG. 1A). The reduction of sphingosine in epithelial cells of the respiratory tract was recapitulated in tracheal and bronchial cells from CF mice (FIG. 1B-D). Next, the inventors tested whether altered sphingosine levels are also present in airway epithelial cells of CerS2 null mice, which display increased levels of ceramide in their tracheal and bronchial epithelial cells (FIGS. 1E, F and 2E), reminiscent of the elevated ceramide levels in CF mice and in CF patients (FIG. 1E, F). These studies revealed a significant reduction of sphingosine levels in bronchial and tracheal epithelial cells of CerS2 null mice (FIG. 1C, D). Acid ceramidase inhalation reduced ceramide levels in bronchial epithelial cells (FIG. 1E, F) of CF and CerS2 null mice, and importantly, increased surface sphingosine in both (FIG. 1C, D).

To quantify sphingosine levels in the epithelia of CF mice, and to confirm the results obtained by immunohistochemical studies using the anti-sphingosine antibody, a number of novel methods were established, including sphingosine and ceramide kinase assays on extracts from freshly isolated tracheal epithelial cells to detect total sphingosine levels, in situ sphingosine and ceramide kinase assays on intact trachea surfaces permitting detection of sphingosine and ceramide exclusively on the luminal surface, and immunoprecipitation of sphingosine upon incubation of the anti-sphingosine antibody with the luminal surface of intact trachea, which also exclusively measures sphingosine on the luminal cell surface. First, freshly isolated tracheal epithelial cells were extracted, dried and subjected to a sphingosine kinase assay (FIG. 2A, left-hand panel), demonstrating an ˜80% reduction of total sphingosine levels in tracheal epithelial cells isolated from CF mice. Next, we performed an in situ sphingosine kinase assay by addition of sphingosine kinase and [³²P]γATP directly to the luminal side of the intact tracheal epithelial cell layer (FIG. 2A, middle panel), revealing an ˜75% reduction of sphingosine on the surface of CF trachea compared to WT controls. The absence of sphingosine on the tracheal surface was confirmed by immunoprecipitation of sphingosine from the luminal membrane of tracheal epithelial cells using the anti-sphingosine antibody coupled to protein L agarose beads, followed by lipid extraction and a sphingosine kinase assay (FIG. 2A, right-hand panel). Addition of acid ceramidase to the surface of the isolated CF trachea prior to the in situ kinase assay normalized sphingosine levels on the tracheal surface (FIG. 2A, middle panel). Incubation of the trachea with 10 μM cytochalasin B (an inhibitor of actin filament polymerization) did not change the amount of sphingosine detected in the in situ sphingosine kinase assay or in the sphingosine immunoprecipitation experiments, excluding the possibility that significant internalization of the kinase or of the antibody occurs during the time of the assay (FIG. 2A, middle and right-hand panels). Incubation with cytochalasin B prevented internalization of P. aeruginosa strain ATCC 27853 into tracheal epithelial cells (data not shown) proving that the drug inhibited cytoskeleton functions in the present experimental set up. Together, these results demonstrate that sphingosine is present on the surface of WT epithelial cells, while almost completely absent on the surface of CF epithelia.

Acid ceramidase inhalation normalized sphingosine and ceramide levels on the surface of CF trachea in vivo (FIG. 2B,C), similar to the results obtained by immunofluorescence using the anti-sphingosine antibody (FIG. 1C-E), reduced ceramide levels in isolated tracheal epithelial cells obtained from CF mice (FIG. 2c, left-hand panel) and on the surface of the trachea (FIG. 2C, middle panel), and importantly, increased surface sphingosine in these cells (FIG. 2B, left-hand panel). Further, sphingosine inhalation restored sphingosine levels on the surface of CF mice tracheal epithelial cells (FIG. 2B, right-hand panel). A ceramide kinase assay was also performed on isolated trachea exposed to acid ceramidase in vitro, showing reduced levels of ceramide and, therefore, confirming the specificity of the reaction (FIG. 2C, right-hand panel).

Cftr has been show to regulate the pH in vesicles of secretory lysosomes by providing Cl⁻-counterions for H⁺ and thus permitting continuous activity of protons pumps. Since proton pumps are present in the plasma membrane, and their activity has been shown to acidify small areas directly on the surface of the plasma membrane, we hypothesized that the pH in small, normally-acidic domains on the cell surface of tracheal epithelial cells also increases in CF. It is important to note that these small areas on the cell membrane are independent of the pH of the airway liquid surface, which has been also shown to be changed in CF. Such an alkalization of small domains on the epithelial surface would shift the ratio of the activities of the acid sphingomyelinase and the acid ceramidase. An increase of the pH from 5.0 to 6.0 results in only a 30-40% reduction of acid sphingomyelinase activity, while acid ceramidase activity is reduced by 80-90% at pH 6. Such an imbalance between the activities of the two enzymes on the cell surface should result in accumulation of ceramide and a marked reduction of sphingosine. Our studies confirm this hypothesis and demonstrate that acidification of CF trachea is sufficient to increase surface sphingosine (FIG. 2D, left-hand panel) and decrease surface ceramide to levels that are similar to those observed in WT trachea (FIG. 2D, right-hand panel), possibly due to increased activity of acid ceramidase.

### Example 2

### Acid Ceramidase or Sphingosine Inhalation Protects Against Pulmonary P. aeruginosa Infection

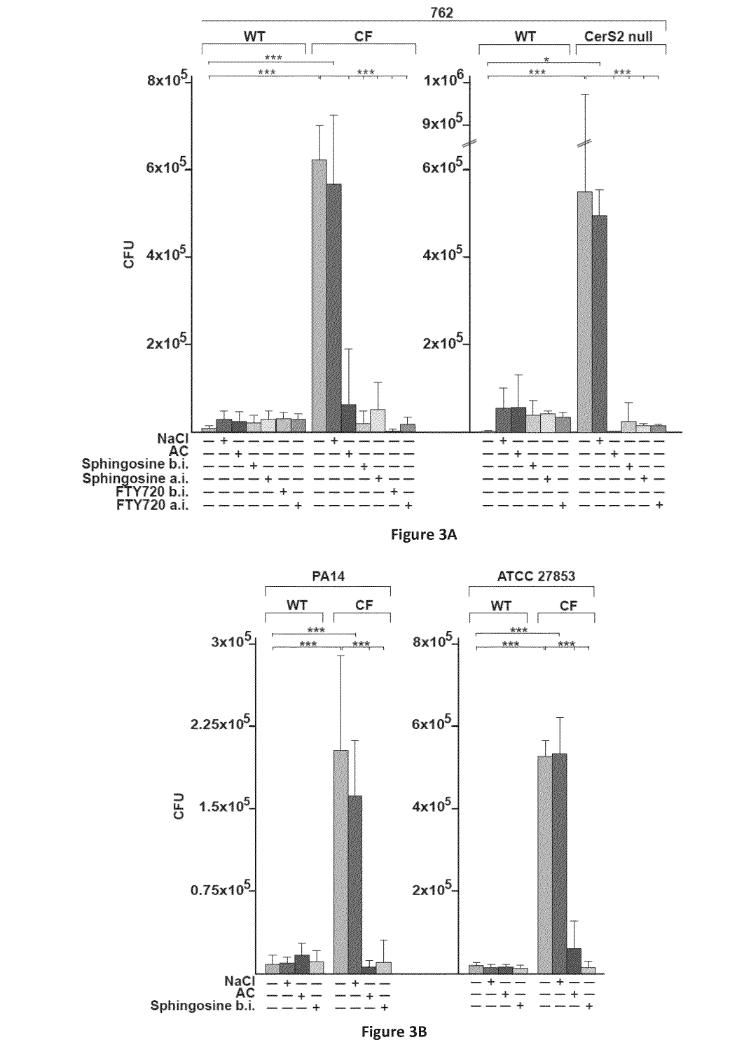

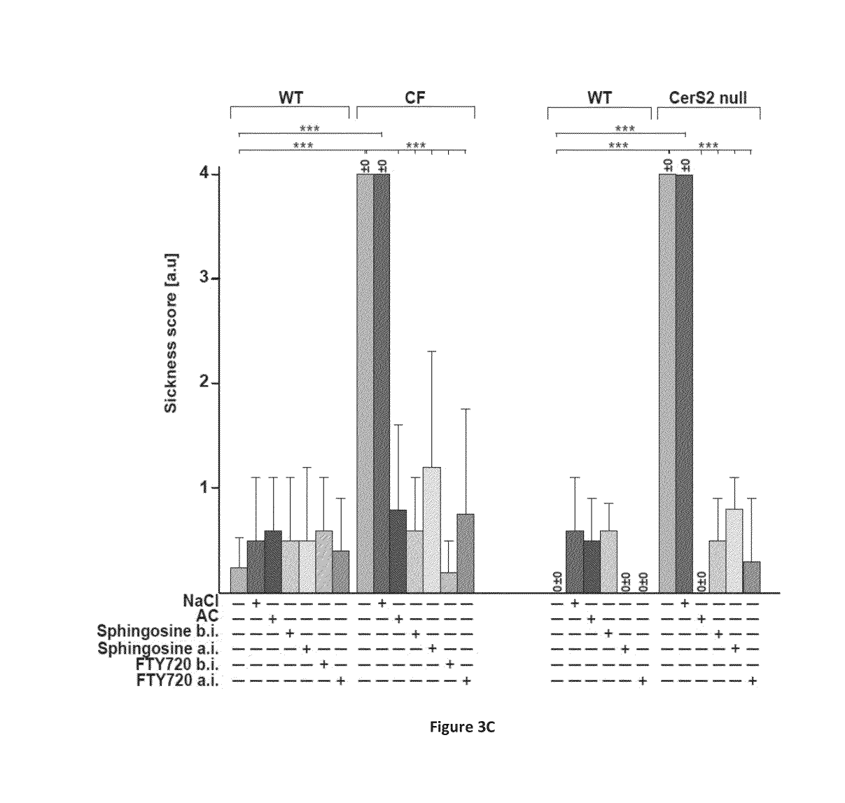

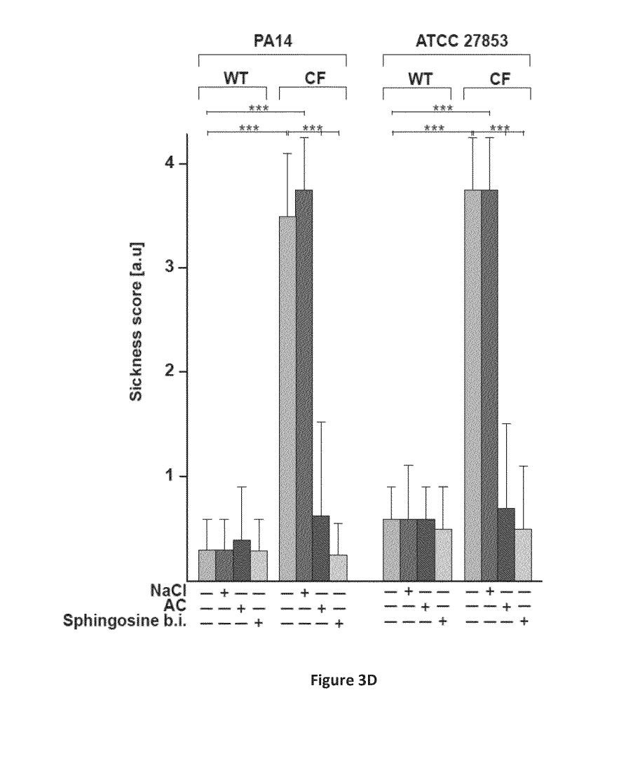

To define the role of sphingosine in the local defense of the respiratory tract against bacterial infection, the inventors first determined the effect of acid ceramidase inhalation on susceptibility to P. aeruginosa infection. Upon intranasal infection, CF and CerS2 null mice displayed a dramatically-increased sensitivity to P. aeruginosa strains 762, PA14 and ATCC 27853, with severe signs of infection and large amounts of bacteria in the lung 3-4 h after infection compared to WT controls, which harbored 10-100-fold less bacteria in their lung (FIG. 3A-D). Acid ceramidase inhalation 1 h prior to P. aeruginosa infection protected both mice from pulmonary infection (FIG. 3A-D).

To determine whether sphingosine is sufficient to prevent or cure pulmonary P. aeruginosa infection, mice were inhaled with P. aeruginosa 1 h prior to or 1 h after infection with sphingosine or with FTY720, a sphingosine analog currently in clinical use for treating multiple sclerosis. Remarkably, sphingosine and FTY720 completely protected CF and CerS2 null mice from P. aeruginosa infection and even eliminated an existing infection with this pathogen (FIG. 3A-D), concomitant with a restoration of surface sphingosine levels (FIG. 2B). Together, this data establishes a novel and critical role of sphingosine on the surface of epithelial cells in the respiratory tract for defense against P. aeruginosa.

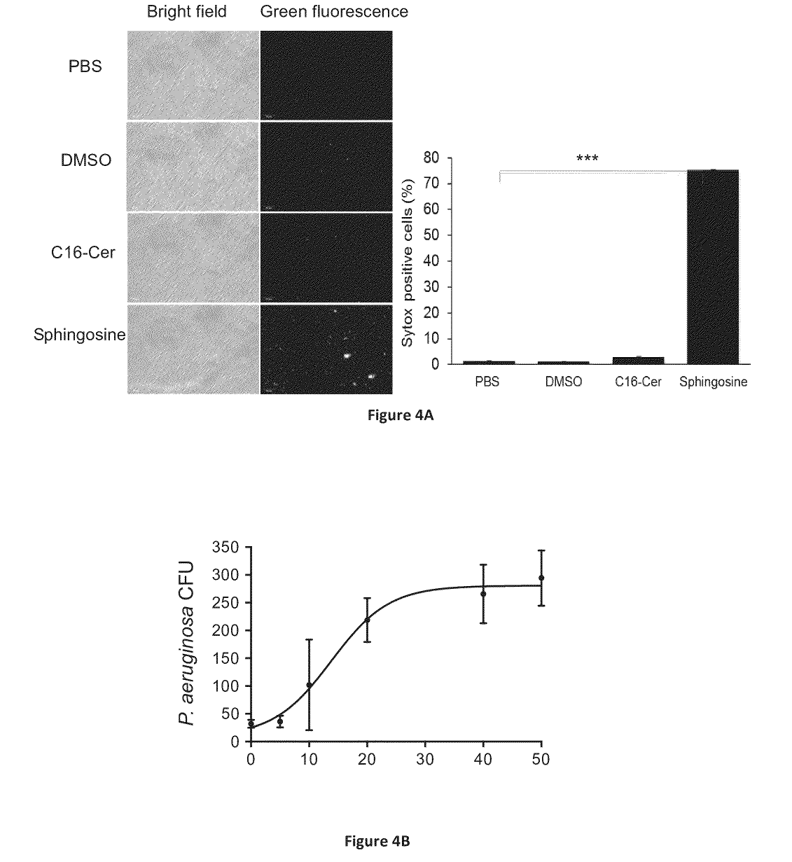

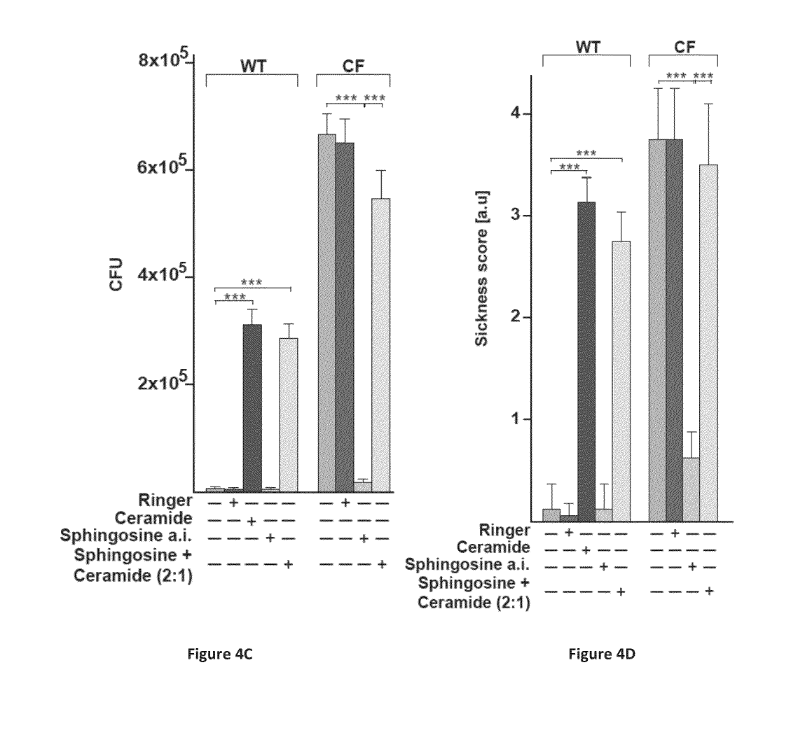

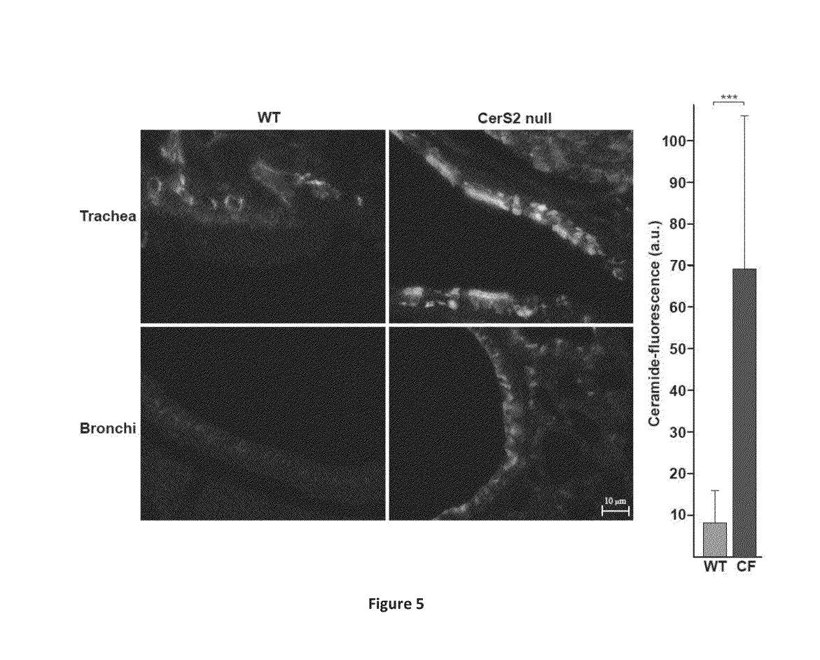

The direct effect of sphingosine and other sphingoid long chain bases (LCBs) on the growth of P. aeruginosa was examined in vitro. Bacteria were incubated with a variety of LCBs and the EC₅₀ values on bacterial growth were determined (Table 1). A number of natural and synthetic LCBs inhibited bacterial growth with EC₅₀ values of 0.3-2.2 μM, including the plant LCB, phytosphingosine (0.9 μM), non-natural stereoisomers of sphingosine and dihydrosphingosine (sphinganine) (0.4-0.8 μM), and LCBs which have recently been discovered to occur at low levels in mammals (Table 1). A number of other LCBs also inhibited bacterial growth with a somewhat higher EC₅₀, including FTY720 (EC₅₀ of 1.9 μM), while other LCBs, including the ceramide synthase inhibitor, fumonisin B1, and sphingosine 1-phosphate were without effect at concentrations as high as 50 μM (Table 1), demonstrating a remarkable specificity in the effect of LCBs on bacterial survival. While these studies were performed with LCBs representing inhibitors with high and low EC₅₀, all drugs were effective killing the bacteria indicating that LCBs even with lower EC₅₀ are effective in vitro opening a wide range structures for drug development. The mechanism of bacterial action involves membrane permeabilization, since incubation of bacteria with Sytox green (a nucleic acid stain) led to a large increase in fluorescence after sphingosine treatment (FIG. 4A).

TABLE 1**
Effect of LCBs on P. aeruginosa growth in vitro. EC₅₀ values were
determined from at least 3 independent experiments. Unless indicated, the LCBs are
C18.

| | | EC50 |
|---|---|---|
| | | (μM) ± |
| Molecule name | Structure | S.D. |
| D-erythro-C20- Sphingosine | 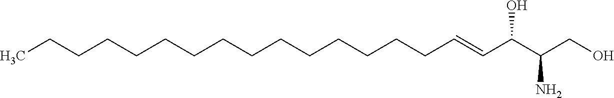 [SMILES: CCCCCCCCCCCCCCC/C=C/[C@H](O)[C@H](N)CO] | 0.3 ± 0.0 |
| L-threo- dihydrosphingosine (Safingol) | 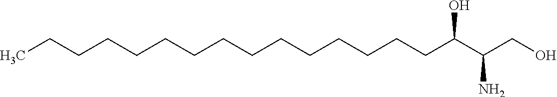 [SMILES: CCCCCCCCCCCCCCC[C@@H](O)[C@H](N)CO] | 0.4 ± 0.0 |
| D-erythro- Sphingosine* | 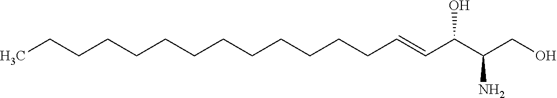 [SMILES: CCCCCCCCCCCCC/C=C/[C@H](O)[C@H](N)CO] | 0.5 ± 0.1 |
| D-threo- dihydrosphingosine | 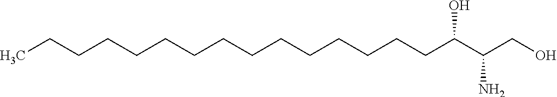 [SMILES: CCCCCCCCCCCCCCC[C@H](O)[C@@H](N)CO] | 0.5 ± 0.1 |
| 3-deoxy-D-erythro- sphingosine | 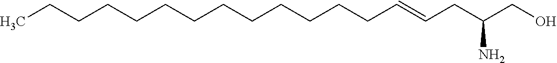 [SMILES: CCCCCCCCCCCCC/C=C/C[C@H](N)CO] | 0.5 ± 0.0 |
| D-erythro- dihydrosphingosine | 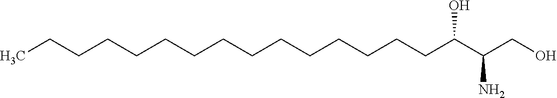 [SMILES: CCCCCCCCCCCCCCC[C@H](O)[C@H](N)CO] | 0.6 ± 0.5 |
| D-threo-sphingosine | 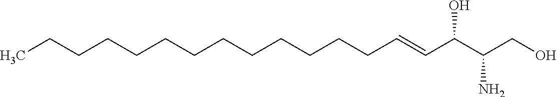 [SMILES: CCCCCCCCCCCCC/C=C/[C@H](O)[C@@H](N)CO] | 0.6 ± 0.1 |
| L-threo-sphingosine | 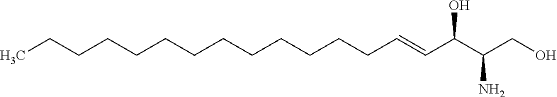 [SMILES: CCCCCCCCCCCCC/C=C/[C@@H](O)[C@H](N)CO] | 0.6 ± 0.2 |
| 1-deoxy-D-erythro- dihydrosphingosine | 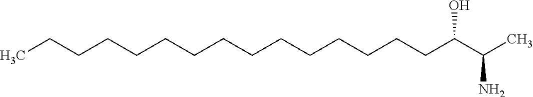 [SMILES: CCCCCCCCCCCCCCC[C@H](O)[C@@H](C)N] | 0.7 ± 0.0 |
| 1-desoxymethyl sphingosine | 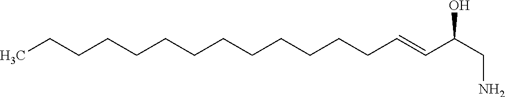 [SMILES: CCCCCCCCCCCCC/C=C/[C@@H](O)CN] | 0.7 ± 0.1 |
| L-erythro-sphingosine | 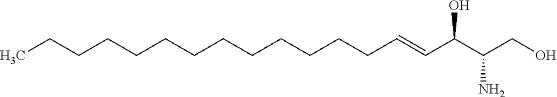 [SMILES: CCCCCCCCCCCCC/C=C/[C@@H](O)[C@@H](N)CO] | 0.8 ± 0.1 |
| L-erythro- dihydrosphingosine | 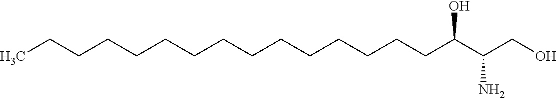 [SMILES: CCCCCCCCCCCCCCC[C@@H](O)[C@@H](N)CO] | 0.8 ± 0.1 |
| 1-deoxy-D-erythro- sphingosine | 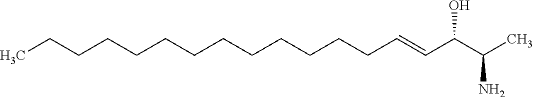 [SMILES: CCCCCCCCCCCCC/C=C/[C@H](O)[C@@H](C)N] | 0.9 ± 0.1 |
| Phyto-sphingosine | 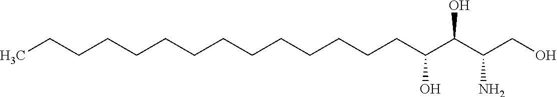 [SMILES: CCCCCCCCCCCCCC[C@@H](O)[C@@H](O)[C@@H](N)CO] | 0.9 ± 0.5 |
| D-erythro-C16- Sphingosine | 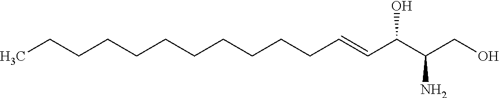 [SMILES: CCCCCCCCCCC/C=C/[C@H](O)[C@H](N)CO] | 1 ± 0.2 |
| FTY720* | 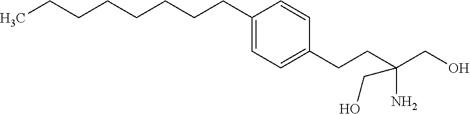 [SMILES: CCCCCCCCc1ccc(CCC(N)(CO)CO)cc1] | 1.9 ± 0.4 |
| Monomethyl D- erythro-Sphingosine | 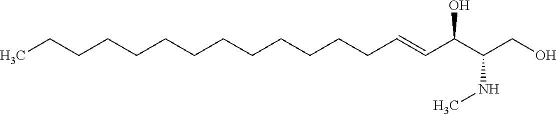 [SMILES: CCCCCCCCCCCCC/C=C/[C@@H](O)[C@H](CO)NC] | 1.9 ± 0.1 |
| Galactosyl Sphingosine (Psychosine) | 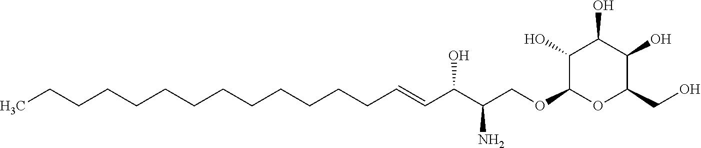 [SMILES: CCCCCCCCCCCCC/C=C/[C@H](O)[C@H](N)CO[C@@H]1O[C@H](CO)[C@H](O)[C@H](O)[C@H]1O] | 2.2 ± 1.4 |

*This LCB was tested in vivo and prevented P. aeruginosa infection in CF- and CerS2 null susceptible strains (FIG. 3)

LCBs that do not inhibit P. aeruginosa growth in vitro are set forth in Table 2.

TABLE 2**
Non-Inhibitors

| | | EC50 |
|---|---|---|
| Molecule name | Structure | (μM) |
| D-erythro-C12- Sphingosine | 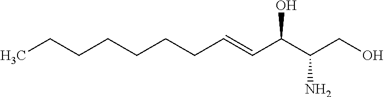 | >50 |
| D-erythro-C14- Sphingosine | 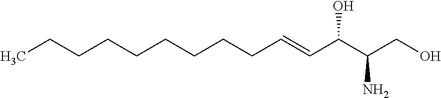 [SMILES: CCCCCCCCC/C=C/[C@H](O)[C@H](N)CO] | >50 |
| N-acetyl-D-erythro- dihydrosphingosine | 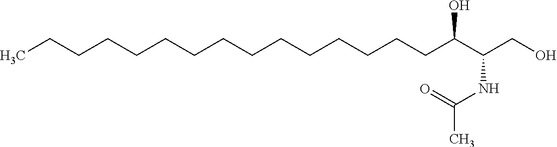 [SMILES: CCCCCCCCCCCCCCC[C@@H](O)[C@H](CO)NC(C)=O] | >50 |
| FB1 | 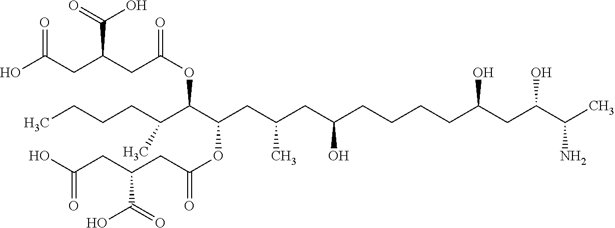 [SMILES: CCCC[C@@H](C)[C@@H](OC(=O)C[C@@H](CC(=O)O)C(=O)O)[C@H](C[C@@H](C)C[C@H](O)CCCC[C@@H](O)C[C@H](O)[C@H](C)N)OC(=O)C[C@@H](CC(=O)O)C(=O)O] | >50 |
| Sphingosine-1-P | 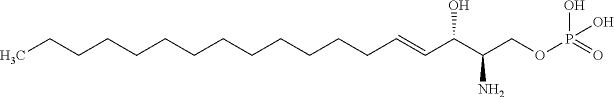 [SMILES: CCCCCCCCCCCCC/C=C/[C@H](O)[C@H](N)COP(=O)(O)O] | >50 |

**For Tables 1 and 2: Measurements were done by growing P. aeruginosa strain 762 from frozen glycerol stock in LB medium, at 37° C. until early logarithmic phase (OD₅₅₀ 0.2). Bacteria were then diluted in PBS to a concentration of 5 × 10³ CFU/ml. Stock solutions (×100) of the various long chain bases (LCBs) were prepared in 7.5% n-Octyl-β-D-Glucopyranoside (OGP-Sigma) following sonication and heating to 40° C. LCBs were added to the bacteria at a final concentration of 0.1-2 μM and in some cases up to 50 μM (in 0.075% OGP), and incubated for 2 hrs at 37° C. Bacteria samples (200 μl) were plated on LB agar plates, incubated overnight at 37° C. and numbers of colonies were counted. EC₅₀ calculations were made by plotting the colonies number in the presence of LCBs vs. vehicle only (OGP) as a function of LCB concentration using Excel software. All experiments were repeated 3 times, except for L-threo-dihydrosphingosine (Safingol), Phyto-sphingosine, and Monomethyl D-erythro-Sphingosine that were repeated 6 times, and D-erythro-Sphingosine that was repeated 9 times.

This data indicates that LCBs analogs act as potent inhibitors of P. aeruginosa infection in vivo and in vitro. Thus, LCBs analogs have a therapeutic use in preventing and treating anti-microbial agents, such as in lung infection. The following non-limiting conclusions may be drawn from the data presented above:

1. Inhibition of P. aeruginosa is positively influenced by the length of the LCB. No inhibition occurs with LCBs containing less than 16 carbons.
2. Stereoisomerism does not seem to play an important role in the in vitro inhibition of P. aeruginosa.
3. The double bond at position 4,5 seems not to play an important role in the in vitro inhibition of P. aeruginosa.
4. The hydroxyl head group is preferable for better inhibition. However, omitting this hydroxyl group or replacing it with sugars also maintains activity. Introduction of phosphate groups (negatively charged) reduces the inhibitory effect of the LCB. Additional hydroxyl groups along the LCB maintains inhibitory activity.
5. Modifications of the amine at position 2 that reduce the overall positive charge of the molecule, also reduce the inhibition effect of the LCB on P. aeruginosa.
6. In general, the ideal LCB has a long carbon chain (18 carbons or longer is preferable), with unmodified hydroxyl and amine groups. Since the bacteria are negatively charged it is most likely that increasing the positive charge of the molecule could be beneficial.

Finally, the relationship between sphingosine and ceramide levels was examined, since previous studies demonstrated that ceramide accumulates in CF and that inhibition of acid sphingomyelinase prevents P. aeruginosa infection of CF mice. Bacteria were incubated in vitro with sphingosine and increasing amounts of C16-ceramide. Even in the presence of 10 μM sphingosine, bacterial growth was restored with 10 μM C16-ceramide and above (FIG. 4B). Moreover, inhalation of CF mice with C16-ceramide and sphingosine at a 1:1 molar ratio abrogated the protective effect of sphingosine and restored susceptibility to P. aeruginosa infection (FIG. 4C,D). Together, these data demonstrate that the balance between ceramide and sphingosine, and their sites of cellular accumulation in tracheal and bronchial epithelial cells, determines the susceptibility of mice to P. aeruginosa infection. Evidence for an anti-microbial effect of sphingosine has been demonstrated in skin infection′ but a role for sphingosine, and other LCBs, has not been shown in lung infection. Further, recombinant acid ceramidase might be a novel enzyme therapy reducing ceramide and elevating sphingosine in the lungs of CF patients.

The in vitro data presented herein, along with the inhalation experiments, suggest a direct effect of LCBs on the bacteria. However, in epithelial cells of healthy airways, sphingosine is located on the luminal surface of the plasma membrane. Thus, sphingosine may kill bacteria by directly interacting with them and inducing formation of pores.

The inventors have identified a large panel of LCBs that kill P. aeruginosa in vitro, paving the way for the development of a wide range of potential novel drugs for anti-microbial inhalation therapy against P. aeruginosa infection in the lung.

### Example 3

### Sphingosine and Other Long Chain Bases (LCBs) Prevent P. aeruginosa Growth In Vitro

Examination of various LCBs in vitro in accordance with the method described in Example 2 (n=3 for each analog) confirmed that the stereoisomerization of the molecules was not an important feature for the inhibitory effect, whereas the electric charge of the LCB was in correlation with the EC₅₀; Positively charged LCBs (e.g: L-threo-sphinganine) were more potent than the neutral ones (e.g: FTY720), while the negatively charged ones (e.g: sphingosine-1-phosphate (S1P) or fumonisin B1) did not inhibit growth of the bacteria at all (Table 3). Since the data indicate that FTY720 is a potent inhibitor in vivo (FIG. 3A) this open a wide range of potential anti-microbial therapy against P. aeruginosa infection in the lung from neutral to very positively charged LCBs.

TABLE 3
The effect of different long chain bases (LCBs) on P. aeruginosa growth in
vitro.

| | | | |
|---|---|---|---|
| | | EC₅₀ | pK (in |
| Name | Formula | [μM] | PBS) |

Strong Inhibitors

| | | | |
|---|---|---|---|
| L-threo- Sphinganine (Safingol) | 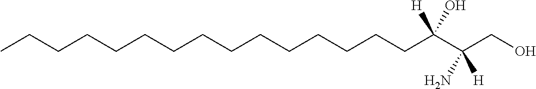 [SMILES: CCCCCCCCCCCCCCC[C@H](O)[C@@H](N)CO] | 0.5-1 | |
| Sphingosine | 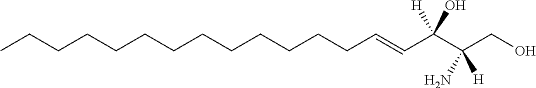 [SMILES: CCCCCCCCCCCCC/C=C/[C@@H](O)[C@@H](N)CO] | 0.5-1 | 6.7^{1,2} |
| L-threo- Sphingosine | 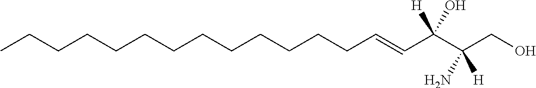 [SMILES: CCCCCCCCCCCCC/C=C/[C@H](O)[C@@H](N)CO] | 0.5-1 | |
| Sphinganine | 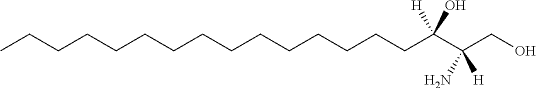 [SMILES: CCCCCCCCCCCCCCC[C@@H](O)[C@@H](N)CO] | 0.5-1 | |

Moderate Inhibitors

| | | | |
|---|---|---|---|
| FTY720 | 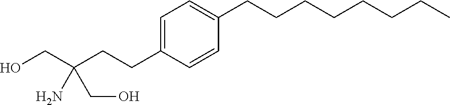 [SMILES: CCCCCCCCc1ccc(CCC(N)(CO)CO)cc1] | 1-5 | 7.8³ |
| Galactosyl Sphingosine (Psychosine) | 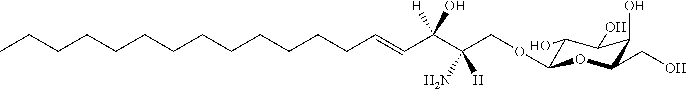 | 1-5 | |
| (R)-FTY- phosphonate | 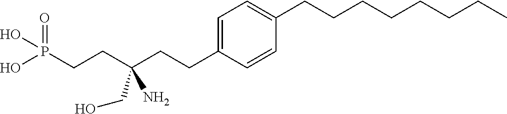 [SMILES: CCCCCCCCc1ccc(CC[C@](N)(CO)CCP(=O)(O)O)cc1] | 5-50 | |

Non Inhibitors

| | | | pKa |
|---|---|---|---|
| | | EC₅₀ | (in |
| Name | Formula | [μM] | PBS) |
| Fumonisin B1 | 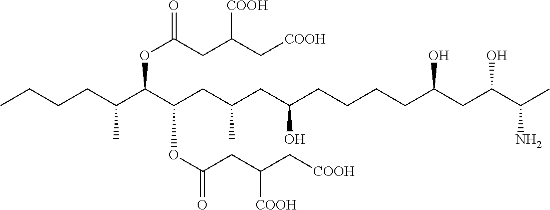 [SMILES: CCCC[C@@H](C)[C@@H](OC(=O)CC(CC(=O)O)C(=O)O)[C@H](C[C@@H](C)C[C@H](O)CCCC[C@@H](O)C[C@H](O)[C@H](C)N)OC(=O)CC(CC(=O)O)C(=O)O] | >50 | >9⁴ |
| Sphingosine-1-P | 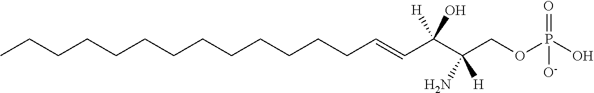 [SMILES: CCCCCCCCCCCCC/C=C/[C@@H](O)[C@@H](N)COP(=O)([O-])O] | >50 | |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1-32. (canceled)

33. A method for preventing or treating a bacterial infection in a subject in need thereof, the method comprising administering to the subject, by inhalation, a therapeutically effective amount of a pharmaceutical composition comprising a sphingoid long chain base (LCB) compound and a pharmaceutically acceptable carrier, wherein the sphingoid LCB compound is represented by the structure of formula (I) or formula (II):
(i) formula (I): 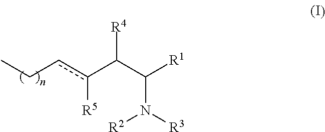
wherein
R¹ is H, CH₃, or —CH₂OR^{a} wherein R^{a} is H, C1-C4 alkyl or a sugar moiety;
R² and R³ are each selected from H or C1-C4 alkyl;
R⁴ and R⁵ are each independently H or OH;
n is an integer between 10 and 16; and [symbol]
is an optional double bond,
with the proviso that the following compounds are excluded:
D-erythro-sphingosine;
4-D-hydroxysphinganine (phytosphingosine);
D-erythro-dihydrosphingosine (D-erythro-sphinganine);
D,L-erythro-dihydrosphingosine (D,L-erythro-sphinganine); and
D,L-threo-dihydrosphingosine (D,L-threo-sphinganine);
and salts, hydrates, solvates, polymorphs, optical isomers, enantiomers, diastereomers, epimers, and mixtures thereof;
(ii) formula (II): 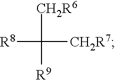
wherein
one of R⁶, R⁷ and R⁸ is NR^{b}R^{c}, and the other two are OR^{d}, wherein R^{b}, R^{c} and R^{d} are each H or C1-C4 alkyl;
R⁹ is a alkyl-phenyl wherein the phenyl is substituted by a C6-C20 alkyl group;
and salts, hydrates, solvates, polymorphs, optical isomers, enantiomers, diastereomers, epimers, and mixtures thereof.

34. The method according to claim 33, wherein the bacteria is a gram-positive bacteria or a gram-negative bacteria.

35. The method of claim 33, wherein said bacterial infection is caused by Pseudomonas, Staphylococcus aureus, Acinetobacter baumanii, Burkholderia species, or Mycobacteria species (typical and atypical).

36. (canceled)

37. The method of claim 33, wherein said bacterial infection is a pulmonary bacterial infection.

38. The method of claim 37, wherein said bacterial infection is in a subject having a lung disorder or disease.

39-43. (canceled)

44. The method according to claim 33, wherein the compound is represented by the structure of formula (I).

45. The method according to claim 44, wherein the compound is a non-natural sphingoid LCB.

46. The method according to claim 45, wherein the compound is selected from non-natural isomers of sphingosine, dihydrosphingosine and phytosphingosine.

47. The method according to claim 44, wherein the compound is selected from a D-threo, L-threo and L-erythro isomer of the compound of formula (I).

48. The method according to claim 44, wherein the sphingoid LCB comprises at least 18 carbons, preferably at least 20 carbons.

49. The method according to claim 44, wherein the sugar is a 5- or 6-carbon monosaccharide, which is preferably in pyranose or furanose form.

50. The method according to claim 44, wherein R¹ is selected from H, CH₂OH, CH₃, CH₂OCH₃, —CH₂—O-galactosyl and —CH₂—O-glucosyl.

51. The method according to claim 44, wherein the compound is represented by the structure of formula III

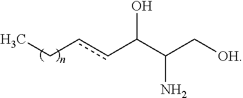

52. The method according to claim 33, wherein the compound is represented by the structure of formula (II).

53. The method according to claim 52, wherein one of R⁶, R⁷ and R⁸ is NH₂, and the other two are OH.

54. The method according to claim 52, wherein the compound of formula (II) is represented by the structure of formula IIa or IIb:

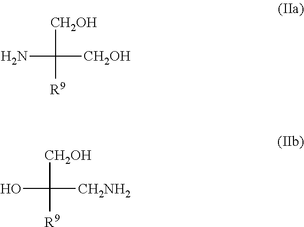

55. The method according to claim 52, wherein R⁹ is an alkyl-phenyl, wherein the phenyl is substituted by a C6-C10 alkyl, preferably by a C8 alkyl.

56. The method according to claim 33, wherein the compound is selected from the group consisting of:
D-erythro-C20-sphingosine;
D-threo-sphingosine;
L-threo-sphingosine;
L-erythro-sphingosine;
D-threo-dihydrosphingosine (D-threo-sphinganine);
L-threo-dihydrosphingosine (L-threo-sphinganine);
L-erythro-dihydrosphingosine (L-erythro-sphinganine);
3-deoxy-D-erythro-sphingosine;
1-deoxy-D-erythro-dihydrosphingosine;
1-desoxymethylsphingosine;
1-deoxy-D-erythro-sphingosine;
D-erythro-C16-sphingosine;
Monomethyl D-erythro-sphingosine;
Galactosyl D-erythro-sphingosine (psychosine);
Glucosyl sphingosine;
2-amino-2-[2-(4-octylphenyl)ethyl]-1,3-propanediol (Fingolimod, FTY720);
2-(aminomethyl)-4-(4-octylphenyl)butane-1,2-diol; and
2-amino-2-(methoxymethyl)-4-(4-octylphenyl)butan-1-ol.

57. A pharmaceutical composition comprising as an active ingredient a sphingoid long chain base (LCB) compound and a pharmaceutically acceptable carrier, wherein the pharmaceutical composition is formulated for inhalation, and wherein the sphingoid LCB compound is represented by the structure of formula (I) or formula (II):
(i) formula (I): 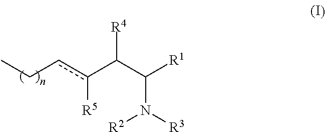
wherein
R¹ is H, CH₃, or —CH₂OR^{a} wherein R^{a} is H, C1-C4 alkyl or a sugar moiety;
R² and R³ are each selected from H or C1-C4 alkyl;
R⁴ and R⁵ are each independently H or OH;
n is an integer between 10 and 16; and [symbol]
is an optional double bond,
with the proviso that the following compounds are excluded:
D-erythro-sphingosine;
4-D-hydroxysphinganine (phytosphingosine);
D-erythro-dihydrosphingosine (D-erythro-sphinganine);
D,L-erythro-dihydrosphingosine (D,L-erythro-sphinganine); and
D,L-threo-dihydrosphingosine (D,L-threo-sphinganine);
and salts, hydrates, solvates, polymorphs, optical isomers, enantiomers, diastereomers, epimers, and mixtures thereof;
(ii) formula (II): 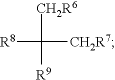
wherein
one of R⁶, R⁷ and R⁸ is NR^{b}R^{c}, and the other two are OR^{d}, wherein R^{b}, R^{c} and R^{d} are each H or C1-C4 alkyl;
R⁹ is a alkyl-phenyl wherein the phenyl is substituted by a C6-C20 alkyl group;
and salts, hydrates, solvates, polymorphs, optical isomers, enantiomers, diastereomers, epimers, and mixtures thereof,
the pharmaceutical composition being identified for use in preventing or treating a bacterial infection.
